(19) United States
(12) Patent Application Publication
(10) Pub. No.: US 2019/0358214 A1
(43) Pub. Date: Nov. 28, 2019

(54) Compositions and Methods for Treating HCV Infection

(71) Applicant: AbbVie Inc., North Chicago, IL (US)

(72) Inventors: Brian Brown, Evanston, IL (US); Hui-Ju Chen, Grayslake, IL (US); David DeGoey, Salem, WI (US); Geoff Halvorsen, Lake Bluff, IL (US); John Hartung, Evanston, IL (US); Howard R. Heyman, Deerfield, IL (US); Nathan Ide, Lindenhurst, IL (US); Dale Kempf, Libertyville, IL (US); Allen C. Krueger, Gurnee, IL (US); Tao Li, Grayslake, IL (US); Tongmei Li, Lake Bluff, IL (US); Steven R. Martinez, North Chicago, IL (US); Christopher C. Marvin, Grayslake, IL (US); John T. Randolph, Libertyville, IL (US); Eric Voight, Pleasant Prairie, WI (US); Rolf Wagner, Antioch, IL (US); Jaclyn Chau, Vancouver (CA)

(73) Assignee: AbbVie Inc., North Chicago, IL (US)

(21) Appl. No.: 16/461,823

(22) PCT Filed: Nov. 13, 2017
(86) PCT No.: PCT/US2017/061283

§ 371 Date: May 17, 2019

Related U.S. Application Data
(60) Provisional application No. 62/423,605, filed on Nov. 17, 2016.
(60) Provisional application No. 62/433,999, filed on Dec. 14, 2016.
(60) Provisional application No. 62/434,589, filed on Dec. 15, 2016.

(51) Int. Cl. A61K 31/454; A61K 9/20; A61K 31/506; A61P 31/14

(52) U.S. Cl. CPC A61K 31/454; A61P 31/14; A61K 31/506; A61K 9/20

(57) ABSTRACT

The present invention features compositions and methods for the treatment of HCV infection. In one embodiment, the compositions of the invention comprise (1) Compound 1 or a pharmaceutically acceptable salt thereof, and (2) Compound 2a or a pharmaceutically acceptable salt thereof. In another embodiment, the compositions of the invention comprise (1) Compound 1 or a pharmaceutically acceptable salt thereof, and (2) a prodrug of Compound 2a (e.g., one of Compounds 2b-2k or Examples 3-1 to 3-10) or a pharmaceutically acceptable salt of said prodrug.

### FIELD OF THE INVENTION

The present invention relates to compositions and methods for treating hepatitis C virus (HCV) infection.

### BACKGROUND OF THE INVENTION

Hepatitis C viral (HCV) infection is a global health problem, with estimated 170 million individuals chronically infected worldwide and at risk of developing liver cirrhosis, hepatocellular carcinoma, or both. Cirrhosis develops after prolonged HCV infection. Complications of cirrhosis include hepatic decompensation (ascites, encephalopathy, variceal hemorrhage, hepatorenal syndrome, or hepatic synthetic dysfunction) and hepatocellular carcinoma.

Treatment of HCV-infected patients could reduce the risk of cirrhosis, decompensation, cancer, and liver-related deaths. Among HCV-infected patients treated with pegylated interferon (pegIFN) and ribavirin (RBV) therapy, achieving a sustained virologic response (SVR) was associated with significant reduction in all-cause death compared to subjects who did not achieve SVR. In addition, the 5-year occurrence of the composite clinical events of death, liver failure, and hepatocellular carcinoma were significantly lower in HCV-infected patients with advanced fibrosis or cirrhosis who achieved SVR versus those without SVR. Patients with SVR following treatment with pegIFN with or without RBV have also shown improvement in liver histology and a reduction in liver-related mortality in several studies. Moreover, patients with compensated cirrhosis who achieve SVR essentially eliminate their subsequent risk of decompensation, may achieve histologic regression, and decrease their risk of hepatocellular carcinoma by two-thirds.

Hepatitis C virus can be classified into 6 major genotypes (GTs) based on sequence divergence of 30% or more. These GTs have evolved differently throughout the world and are divided into several subgenotypes. HCV GT1 is the most common worldwide; however, HCV GT1 subtypes vary by geographic region. HCV GT1 is the most common GT in North America and Europe.

Until recently, treatment for HCV infection included pegIFN and RBV coadministered with one of the direct-acting antivirals (DAAs) telaprevir, boceprevir, and sofosbuvir. These therapies had significant limitations including suboptimal response rates in key patient populations, significant side effects, and prolonged treatment durations (up to 48 weeks). New DAAs that can be used without interferon have recently been approved in US, the European Union (EU), and other countries transforming the treatment of chronic HCV into a relatively short, well-tolerated course of therapy.

### DETAILED DESCRIPTION OF THE INVENTION

In one aspect, the present invention features a pharmaceutical composition comprising (1)

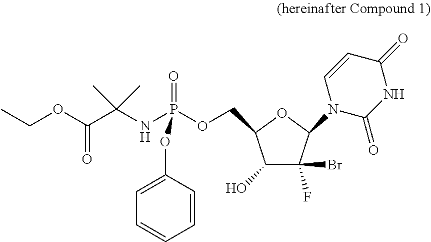 [SMILES: CCOC(=O)C(C)(C)N[P@](=O)(OC[C@H]1O[C@@H](n2ccc(=O)[nH]c2=O)[C@@](F)(Br)[C@@H]1O)Oc1ccccc1]

or a pharmaceutically acceptable salt thereof, and (2)

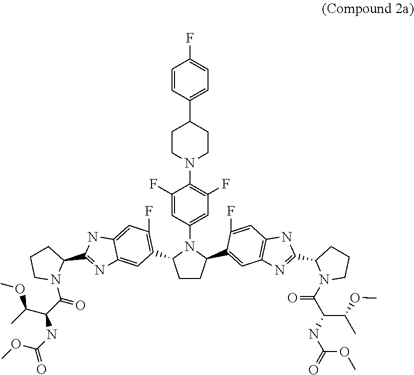

or a pharmaceutically acceptable salt thereof Preferably, the pharmaceutical composition is an orally administered dosage form. More preferably, the pharmaceutical composition is an orally administered tablet.

Compound 1 is a HCV polymerase inhibitor described in U.S. patent application Ser. No. 15/254,342, filed Sep. 1, 2016. Compound 2a is a HCV NSSA inhibitor described in U.S. Pat. No. 8,937,150, and is also known as pibrentasvir.

In another aspect, the present invention features a pharmaceutical composition comprising (1) Compound 1 or a pharmaceutically acceptable salt thereof, and (2) a prodrug of Compound 2a or a pharmaceutically acceptable salt of said prodrug. Preferably, the pharmaceutical composition is an orally administered dosage form. More preferably, the pharmaceutical composition is an orally administered tablet. In this aspect, the prodrug of Compound 2a can be, for example, Compounds 2b. In this aspect, the prodrug of Compound 2a can be, for example, Compounds 2c. In this aspect, the prodrug of Compound 2a can be, for example, Compounds 2d. In this aspect, the prodrug of Compound 2a can be, for example, Compounds 2e. In this aspect, the prodrug of Compound 2a can be, for example, Compounds 2f In this aspect, the prodrug of Compound 2a can be, for example, Compounds 2g. In this aspect, the prodrug of Compound 2a can be, for example, Compounds 2h. In this aspect, the prodrug of Compound 2a can be, for example, Compounds 2i. In this aspect, the prodrug of Compound 2a can be, for example, Compounds 2j. In this aspect, the prodrug of Compound 2a can be, for example, Compounds 2k. In this aspect, the prodrug of Compound 2a can be, for example, the compound of Example 3-1. In this aspect, the prodrug of Compound 2a can be, for example, the compound of Example 3-2. In this aspect, the prodrug of Compound 2a can be, for example, the compound of Example 3-3. In this aspect, the prodrug of Compound 2a can be, for example, the compound of Example 3-4. In this aspect, the prodrug of Compound 2a can be, for example, the compound of Example 3-5. In this aspect, the prodrug of Compound 2a can be, for example, the compound of Example 3-6. In this aspect, the prodrug of Compound 2a can be, for example, the compound of Example 3-7. In this aspect, the prodrug of Compound 2a can be, for example, the compound of Example 3-8. In this aspect, the prodrug of Compound 2a can be, for example, the compound of Example 3-9. In this aspect, the prodrug of Compound 2a can be, for example, the compound of Example 3-10. Compounds 2b-2k and Examples 3-1 to 3-10 are described below.

As used herein, if in one aspect, the invention features a pharmaceutical composition or method having feature A selected from feature A₁, feature A₂, feature A₃, and so on, and in another aspect, the invention features a pharmaceutical composition or method having feature B selected from feature B₁, feature B₂, feature B₃, and so on, then the invention encompasses a pharmaceutical composition or method having both feature A and a feature B in other words, the invention encompasses a pharmaceutical composition or method having both feature A₁ and feature B₁; the invention also encompasses a pharmaceutical composition or method having both feature A₁ and feature B₂; the invention also encompasses a pharmaceutical composition or method having both feature A₁ and feature B₃; the invention also encompasses a pharmaceutical composition or method having both feature A₂ and feature B₁; the invention also encompasses a pharmaceutical composition or method having both feature A₂ and feature B₂; the invention also encompasses a pharmaceutical composition or method having both feature A₂ and feature B₃; the invention also encompasses a pharmaceutical composition or method having both feature A₃ and feature B₁; the invention also encompasses a pharmaceutical composition or method having both feature A₃ and feature B₂; the invention also encompasses a pharmaceutical composition or method having both feature A₃ and feature B₃; and so on.

As used herein, prodrug refers to a derivative of a compound, which has an additional chemically or metabolically cleavable group(s) on the compound and becomes, by solvolysis or under physiological conditions, the compound. A prodrug of a compound can be formed in a conventional manner by reaction of a functional group of the compound (such as an amino, hydroxy, carboxy or phosphate group). Prodrugs often offer advantages of solubility, tissue compatibility, or delayed release in mammals (see, Bungard, H., DESIGN OF PRODRUGS, pp. 7-9, 21-24, Elsevier, Amsterdam 1985). Prodrugs include acid derivatives well known to practitioners of the art, such as, for example, esters prepared by reaction of the parent acidic compound with a suitable alcohol, or amides prepared by reaction of the parent acid compound with a suitable amine. Examples of prodrugs include, but are not limited to, acetate, formate, benzoate or other acylated derivatives of alcohol or amine functional groups within the parent compounds, or phosphate esters of the parent compounds. Some prodrugs are aliphatic or aromatic esters derived from acidic groups on a parent compound. Some prodrugs are aliphatic or aromatic esters of hydroxyl or amino groups on the parent compounds. Phosphate prodrugs of hydroxyl groups are preferred prodrugs.

In another aspect, the present invention features a pharmaceutical composition comprising (1) Compound 1 or a pharmaceutically acceptable salt thereof, and (2)

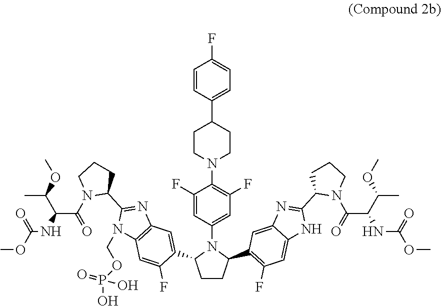

or a pharmaceutically acceptable salt thereof. Preferably, the pharmaceutical composition is an orally administered dosage form. More preferably, the pharmaceutical composition is an orally administered tablet.

In another aspect, the present invention features a pharmaceutical composition comprising (1) Compound 1 or a pharmaceutically acceptable salt thereof, and (2)

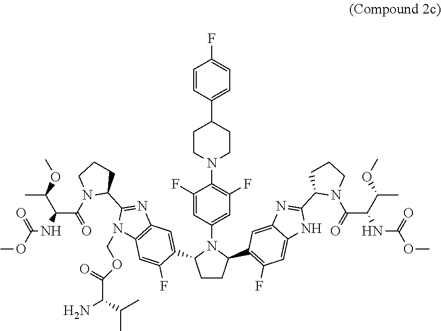

or a pharmaceutically acceptable salt thereof. Preferably, the pharmaceutical composition is an orally administered dosage form. More preferably, the pharmaceutical composition is an orally administered tablet.

In another aspect, the present invention features a pharmaceutical composition comprising (1) Compound 1 or a pharmaceutically acceptable salt thereof, and (2) Compound 2d or a pharmaceutically acceptable salt thereof, wherein Compound 2d is selected from Example 3-1, 3-2, 3-3, 3-4, 3-5, 3-6, 3-7 3-8, 3-9 or 3-10, as described below. Preferably, the pharmaceutical composition is an orally administered dosage form. More preferably, the pharmaceutical composition is an orally administered tablet.

In another aspect, the present invention features a pharmaceutical composition comprising (1) Compound 1 or a pharmaceutically acceptable salt thereof, and (2) Compound 2e or a pharmaceutically acceptable salt thereof, Compound 2e having Formula I:

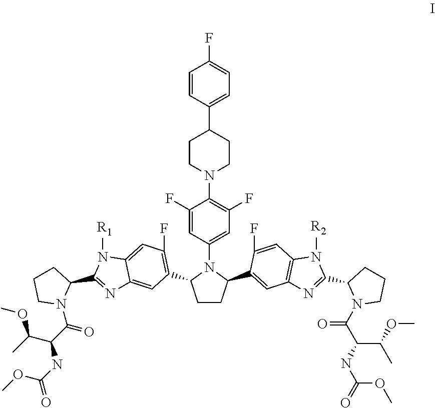

wherein:

R₁ is H or -L₁-R_{A};

R₂ is H or -L₂-Rᵤ;

R_{A} is

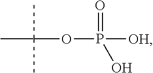

—O(CO)—C₁-C₆alkyl, or —OC(O)-L₃-NH₂;

R_{B} is

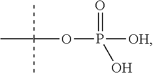

—O(CO)C—C₁-C₆alkyl, or OC(O)-L₄-NH₂; and

L₁, L₂, L₃ and L₄ are each independently C₁-C₆alkylene, provided that R₁ and R₂ cannot be both H. Preferably, L₁ and L₂ are —CH₃—. Preferably, the pharmaceutical composition is an orally administered dosage form. More preferably, the pharmaceutical composition is an orally administered tablet.

As used herein, any alkyl and alkylene can be either a straight or branched saturated hydrocarbyl chain, or a straight or branched unsaturated hydrocarbyl chain.

In another aspect, the present invention features a pharmaceutical composition comprising (1) Compound 1 or a pharmaceutically acceptable salt thereof, and (2) Compound 2f or a pharmaceutically acceptable salt thereof, Compound 2f having Formula II:

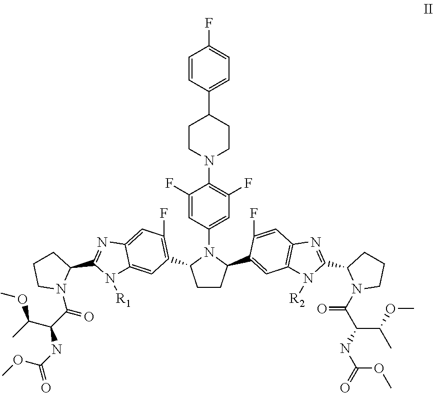

wherein:

R₁ is H or -L₁-R_{A};

R₂ is H or -L₂-R_{B};

R_{A} is

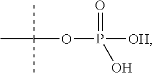

—O(CO)—C₁-C₆alkyl, or —OC(O)-L₃-NH₂;

R_{B} is

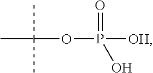

—O(CO)—C₁-C₆alkyl, or —OC(O)-L₄-NH₂; and

L₁, L₂, L₃ and L₄ are each independently C₁-C₆alkylene, provided that R₁ and R₂ cannot be both H. Preferably, L₁ and L₂ are —CH₃—. Preferably, the pharmaceutical composition is an orally administered dosage form. More preferably, the pharmaceutical composition is an orally administered tablet.

In another aspect, the present invention features a pharmaceutical composition comprising (1) Compound 1 or a pharmaceutically acceptable salt thereof, and (2) Compound 2g or a pharmaceutically acceptable salt thereof, Compound 2g having Formula III:

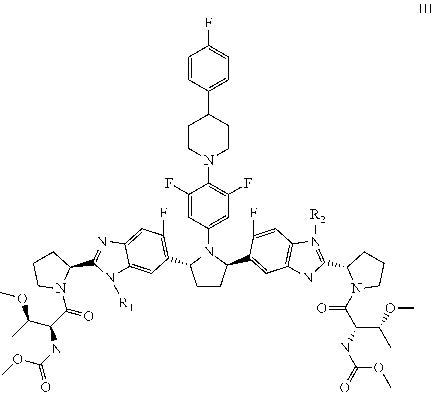

wherein:

R₁ is H or -L₁-R_{A};

R₂ is H or -L₂-R_{B};

R_{A} is

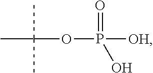

—O(CO)—C₁-C₆alkyl, or —OC(O)-L₃-NH₂;

R_{B} is

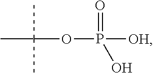

—O(CO)—C₁-C₆alkyl, or —OC(O)-L₄-NH₂; and

L₁, L₂, L₃ and L₄ are each independently C₁-C₆alkylene, provided that R₁ and R₂ cannot be both H. Preferably, L₁ and L₂ are —CH₃—. Preferably, the pharmaceutical composition is an orally administered dosage form. More preferably, the pharmaceutical composition is an orally administered tablet.

In another aspect, the present invention features a pharmaceutical composition comprising (1) Compound 1 or a pharmaceutically acceptable salt thereof, and (2) Compound 2h or a pharmaceutically acceptable salt thereof, Compound 2h having Formula IV:

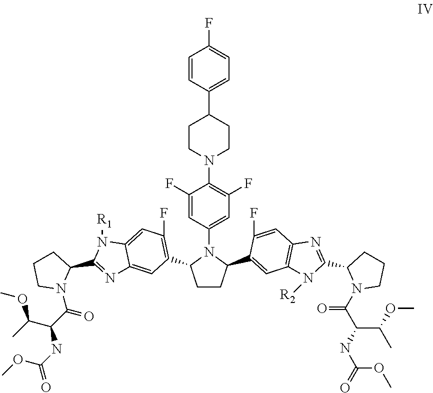

wherein:

R₁ is H or -L₁-R_{A};

R₂ is H or -L₂-R_{B};

R_{A} is

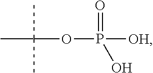

—O(CO)—C₁-C₆alkyl, or —OC(O)-L₃-NH₂;

R_{B} is

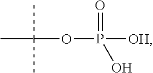

—O(CO)—C₁-C₆alkyl, or —OC(O)—L₄—NH₂; and

L₁, L₂, L₃ and L₄ are each independently C₁-C₆alkylene, provided that R₁ and R₂ cannot be both H. Preferably, L₁ and L₂ are —CH₃—. Preferably, the pharmaceutical composition is an orally administered dosage form. More preferably, the pharmaceutical composition is an orally administered tablet.

In another aspect, the present invention features a pharmaceutical composition comprising (1) Compound 1 or a pharmaceutically acceptable salt thereof, and (2) Compound 2i or a pharmaceutically acceptable salt thereof, Compound 2i having Formula V:

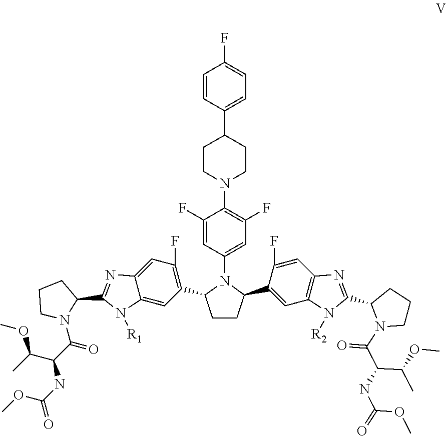

wherein R₁ is either H or selected from Table 1, R₂ is H or selected from Table 1, provided that R₁ and R₂ cannot be both H.

TABLE 1

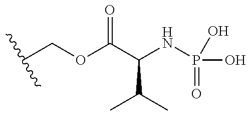 [SMILES: *COC(=O)[C@@H](NP(=O)(O)O)C(C)C]

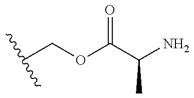 [SMILES: *COC(=O)[C@H](C)N]

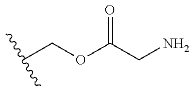 [SMILES: *COC(=O)CN]

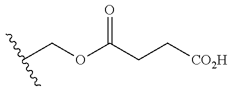 [SMILES: *COC(=O)CCC(=O)O]

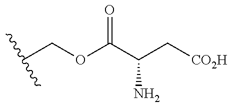 [SMILES: *COC(=O)[C@@H](N)CC(=O)O]

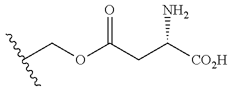 [SMILES: *COC(=O)C[C@H](N)C(=O)O]

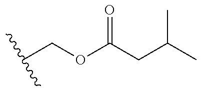 [SMILES: *COC(=O)CC(C)C]

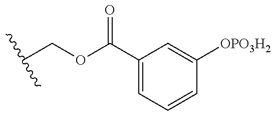

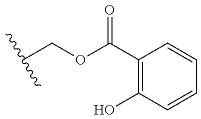 [SMILES: *COC(=O)c1ccccc1O]

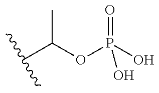 [SMILES: *C(C)OP(=O)(O)O]

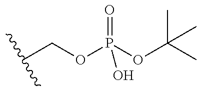 [SMILES: *COP(=O)(O)OC(C)(C)C]

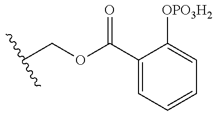

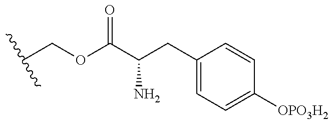

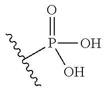 [SMILES: *P(=O)(O)O]

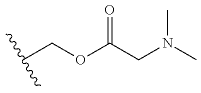 [SMILES: *COC(=O)CN(C)C]

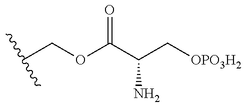

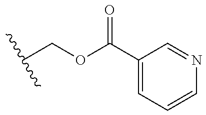 [SMILES: *COC(=O)c1cccnc1]

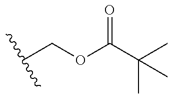 [SMILES: *COC(=O)C(C)(C)C]

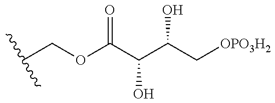

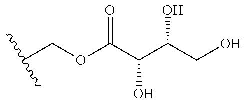 [SMILES: *COC(=O)[C@@H](O)[C@H](O)CO]

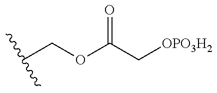

Preferably, the pharmaceutical composition is an orally administered dosage form. More preferably, the pharmaceutical composition is an orally administered tablet.

In another aspect, the present invention features a pharmaceutical composition comprising (1) Compound 1 or a pharmaceutically acceptable salt thereof, and (2) Compound 2j or a pharmaceutically acceptable salt thereof, Compound 2j having Formula VI:

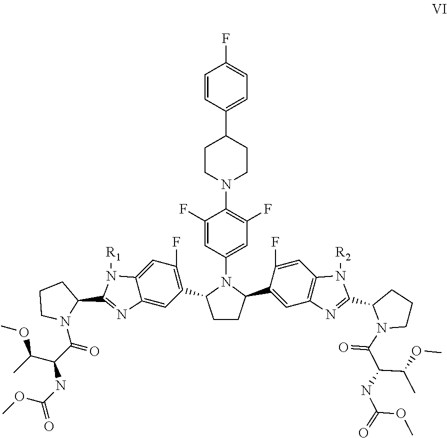

wherein R₁ is either H or selected from Table 1, R₂ is H or selected from Table 1, provided that R₁ and R₂ cannot be both H. Preferably, the pharmaceutical composition is an orally administered dosage form. More preferably, the pharmaceutical composition is an orally administered tablet.

In another aspect, the present invention features a pharmaceutical composition comprising (1) Compound 1 or a pharmaceutically acceptable salt thereof, and (2) Compound 2k or a pharmaceutically acceptable salt thereof, Compound 2k having Formula VII:

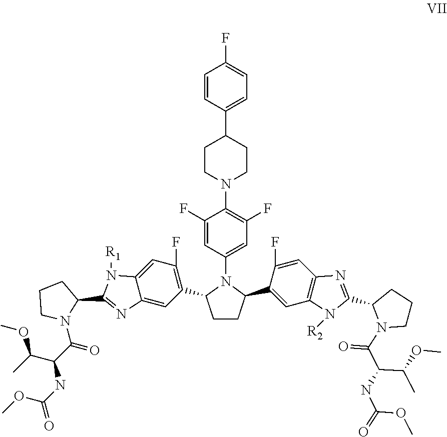

wherein R₁ is either H or selected from Table 1, R₂ is H or selected from Table 1, provided that R₁ and R₂ cannot be both H. Preferably, the pharmaceutical composition is an orally administered dosage form. More preferably, the pharmaceutical composition is an orally administered tablet.

Any pharmaceutical composition of the present invention described herein typically includes a pharmaceutically acceptable carrier or excipient. Non-limiting examples of suitable pharmaceutically acceptable carriers/excipients include sugars (e.g., lactose, glucose or sucrose), starches (e.g., corn starch or potato starch), cellulose or its derivatives (e.g., sodium carboxymethyl cellulose, ethyl cellulose or cellulose acetate), oils (e.g., peanut oil, cottonseed oil, safflower oil, sesame oil, olive oil, corn oil or soybean oil), glycols (e.g., propylene glycol), buffering agents (e.g., magnesium hydroxide or aluminum hydroxide), agar, alginic acid, powdered tragacanth, malt, gelatin, talc, cocoa butter, pyrogen-free water, isotonic saline, Ringer's solution, ethanol, or phosphate buffer solutions. Lubricants, coloring agents, releasing agents, coating agents, sweetening, flavoring or perfuming agents, preservatives, or antioxidants can also be included in a pharmaceutical composition of the present invention.

Various additives can also be included in a pharmaceutical composition of the invention. For instance, at least one additive selected from flow regulators, binders, lubricants, fillers, disintegrants, plasticizers, colorants, or stabilizers may be included. Disintegrants promote a rapid disintegration of the compact in the stomach and keeps the liberated granules separate from one another. Non-limiting examples of suitable disintegrants are cross-linked polymers such as cross-linked polyvinyl pyrrolidone, cross-linked sodium carboxymethylcellulose or sodium croscarmellose. Non-limiting examples of suitable fillers (also referred to as bulking agents) are lactose monohydrate, calcium hydrogenphosphate, microcrystalline cellulose (e.g., Avicell), silicates, in particular silicium dioxide, magnesium oxide, talc, potato or corn starch, isomalt, or polyvinyl alcohol. Non-limiting examples of suitable flow regulators include highly dispersed silica (e.g., colloidal silica such as Aerosil), and animal or vegetable fats or waxes. Non-limiting examples of suitable lubricants include polyethylene glycol (e.g., having a molecular weight of from 1000 to 6000), magnesium and calcium stearates, sodium stearyl fumarate, and the like. Non-limiting examples of stabilizers include antioxidants, light stabilizers, radical scavengers, or stabilizers against microbial attack.

A pharmaceutical composition of the present invention can be formulated based on their routes of administration using methods well known in the art. For example, a sterile injectable preparation can be prepared as a sterile injectable aqueous or oleagenous suspension using suitable dispersing or wetting agents and suspending agents. Suppositories for rectal administration can be prepared by mixing drugs with a suitable nonirritating excipient such as cocoa butter or polyethylene glycols which are solid at ordinary temperatures but liquid at the rectal temperature and will therefore melt in the rectum and release the drugs. Solid dosage forms for oral administration can be capsules, tablets, pills, powders or granules. In such solid dosage forms, the active compounds can be admixed with at least one inert diluent such as sucrose lactose or starch. Solid dosage forms may also comprise other substances in addition to inert diluents, such as lubricating agents. In the case of capsules, tablets and pills, the dosage forms may also comprise buffering agents. Tablets and pills can additionally be prepared with enteric coatings. Liquid dosage forms for oral administration can include pharmaceutically acceptable emulsions, solutions, suspensions, syrups or elixirs containing inert diluents commonly used in the art. Liquid dosage forms may also comprise wetting, emulsifying, suspending, sweetening, flavoring, or perfuming agents. The pharmaceutical compositions of the present invention can also be administered in the form of liposomes, as described in U.S. Pat. No. 6,703,403. Formulation of drugs that are applicable to the present invention is generally discussed in, for example, Hoover, John E., REMINGTON'S PHARMACEUTICAL SCIENCES (Mack Publishing Co., Easton, Pa.: 1975), and Lachman, L., eds., PHARMACEUTICAL DOSAGE FORMS (Marcel Decker, New York, N.Y., 1980).

In one aspect, Compound 2a, 2b, 2c, 2d, 2e, 2f, 2g, 2h, 2i, 2j or 2k in any pharmaceutical composition of the invention as described herein is formulated in a solid dispersion, preferably in an amorphous solid dispersion. The solid dispersion (preferably the amorphous solid dispersion) can further comprise a pharmaceutically acceptable hydrophilic polymer and, optionally, a pharmaceutically acceptable surfactant. Suitable solid dispersion technology includes, but is not limited to, melt-extrusion, spray-drying, co-precipitation, freeze drying, or other solvent evaporation techniques, with melt-extrusion and spray-drying being preferred.

Any pharmaceutical composition of the invention as described herein can also comprise another DAA known in the art, such as a HCV protease inhibitor.

In one aspect, any pharmaceutical composition of the invention as described hereinabove further comprises Compound 3 or a pharmaceutically acceptable salt thereof, wherein Compound 3 is glecaprevir

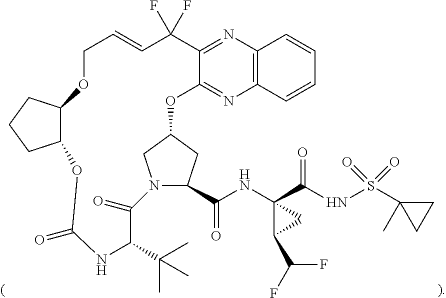 [SMILES: CC(C)(C)[C@@H]1NC(=O)O[C@@H]2CCC[C@H]2OC/C=C/C(F)(F)c2nc3ccccc3nc2O[C@@H]2C[C@@H](C(=O)N[C@]3(C(=O)NS(=O)(=O)C4(C)CC4)C[C@H]3C(F)F)N(C2)C1=O]

In one aspect, the present invention features methods of treating HCV infection using a pharmaceutical composition of the invention. Any pharmaceutical composition described herein can be used in these methods. The methods comprise administering the pharmaceutical composition to a HCV patient. Preferably, these methods do not include the use of interferon or ribavirin. More preferably, these methods do not include the use of interferon or ribavirin, and last for 4, 5, 6, 7, 8, 9, 10, 11 or 12 weeks. As used herein, if a method lasts for n weeks, it means that the treatment is completed at the end of the n weeks.

In another embodiment, these methods do not include the use of interferon or ribavirin, and last for 4 weeks.

In another embodiment, these methods do not include the use of interferon or ribavirin, and last for 6 weeks.

In one embodiment, these methods do not include the use of interferon or ribavirin, and last for 8 weeks.

In another embodiment, these methods do not include the use of interferon or ribavirin, and last for 10 weeks.

In another embodiment, these methods do not include the use of interferon or ribavirin, and last for 12 weeks.

The patient that is treated by any method described herein can be a patient infected with HCV genotype 1. The patient that is treated by any method described herein can be a patient infected with HCV genotype 2. The patient that is treated by any method described herein can be a patient infected with HCV genotype 3 The patient that is treated by any method described herein can be a patient infected with HCV genotype 4. The patient that is treated by any method described herein can be a patient infected with HCV genotype 5. The patient that is treated by any method described herein can be a patient infected with HCV genotype 6. The patient that is treated by any method described herein can be a treatment-nave patient. The patient that is treated by any method described herein can be a treatment-experienced patient. The patient that is treated by any method described herein can be a patient without cirrhosis. The patient that is treated by any method described herein can be a patient with compensated cirrhosis.

The present invention also features methods of making Compound 1 as shown in Scheme I, wherein R is a alkyl, —C(O)N(H)-alkyl or alike.

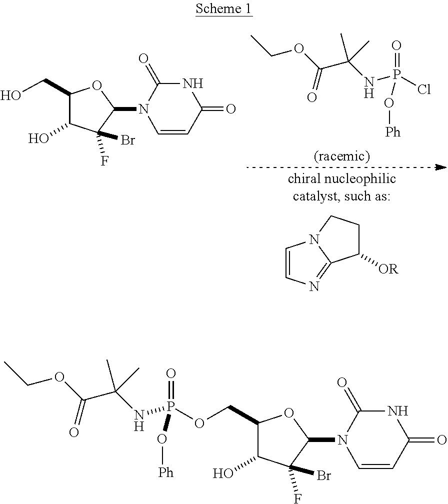

The methods involve resolution of racemic chlorophosphoramidating reagents with a nucleophilic catalyst. A library of catalysts for this transformation as shown in Scheme I is available, such as chiral bicyclic imidazoles (e.g., a bicyclic imidazole ethyl ether such as

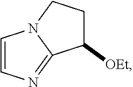 [SMILES: CCO[C@@H]1CCn2ccnc21]

or a chiral bicyclic imidazole t-butylcarbamate such as

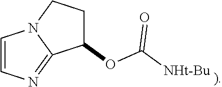

These catalysts, as well as other suitable nucleophilic catalysts (e.g., pyridine-based) can help resolve a racemic aminoisoburic acid (AIB) ethyl ester chlorophosphoramidate, allowing access to compounds such as Compound 1 enriched in the preferred diastereomer, controlled by the chiral catalyst.

As described in Example 41 of U.S. patent application Ser. No. 15/254,342 (filed Sep. 1, 2016), Compound 1 showed significantly improved active triphosphate profiles as compared to sofosbuvir. This was believed to be attributed to the unique prodrug moiety in Compound 1, which is different from that in sofosbuvir. See paragraphs [0017]. [0018] and [0019] of U.S. patent application Ser. No. 15/254,342. In 4-day repeat dose dog liver biopsy study, Compound 1 dosed orally at 1 mg/kg delivered the similar liver triphosphate levels as sofosbuvir dosed orally at 5 mg/kg. Further modeling provided that 75 mg Compound 1 once daily dosing in human patients would match the liver triphosphate Cₘₐₓ achieved by 400 mg sofosbuvir once daily dosing, that 80 mg Compound 1 once daily dosing in human patients would match the liver triphosphate AUC₀₋₂₄ achieved by 400 mg sofosbuvir once daily dosing, and that 16 mg Compound 1 once daily dosing in human patients would maintain Cₘᵢₙ above GT1b polymerase EC₅₀. The model that was used to translate dog study results to human liver pharmacokinetics was validated by sofosbuvir data.

Accordingly, in any method described herein, Compound 1 (or a pharmaceutically acceptable salt thereof) preferably is dosed at 60-100 mg once daily.

In any method described herein, Compound 1 (or a pharmaceutically acceptable salt thereof) more preferably is dosed at 70, 75, 80, 85, 90, 95 or 100 mg once daily.

In any method described herein, Compound 1 (or a pharmaceutically acceptable salt thereof) highly preferably is dosed at 80 mg once daily.

In any method described herein, Compound 1 (or a pharmaceutically acceptable salt thereof) highly preferably is dosed at 90 mg once daily.

In any method described herein, Compound 1 (or a pharmaceutically acceptable salt thereof) highly preferably is dosed at 100 mg once daily.

Likewise, in any pharmaceutical composition described herein, the composition preferably contains 60-100 mg Compound 1 (or a pharmaceutically acceptable salt thereof).

In any pharmaceutical composition described herein, the composition more preferably contains 70, 75, 80, 85, 90, 95 or 100 mg Compound 1 (or a pharmaceutically acceptable salt thereof).

In any pharmaceutical composition described herein, the composition highly preferably contains 80 mg Compound 1 (or a pharmaceutically acceptable salt thereof).

In any pharmaceutical composition described herein, the composition highly preferably contains 90 mg Compound 1 (or a pharmaceutically acceptable salt thereof).

In any pharmaceutical composition described herein, the composition highly preferably contains 100 mg Compound 1 (or a pharmaceutically acceptable salt thereof).

Lower dosing of Compound 1 (e.g., 80 mg once daily) allows the treatment of HCV patients with end stage renal disease. Accordingly, in any method described herein (e.g., when 80 mg Compound 1 once daily is used), the patient being treated can have end stage renal disease.

Lower dosing of Compound 1 (e.g., 80 mg once daily) also allows the treatment of HCV patients with severe renal impairment. Accordingly, in any method described herein (e.g., when 80 mg Compound 1 once daily is used), the patient being treated can have severe renal impairment.

It should be understood that the above-described embodiments and the following examples are given by way of illustration, not limitation. Various changes and modifications within the scope of the present invention will become apparent to those skilled in the art from the present description.

### EXAMPLE 1

### Ethyl 2-(((S)-(((2R,3R,4S,5R)-4-bromo-5-(2,4-dioxo-3,4-dihydropyrimidin-1(2H)-yl)-4-fluoro-3-hydroxytetrahydrofuran-2-yl)methoxy)(phenoxy)phosphoryl)amino)-2-methylpropanoate

### EXAMPLE 1A

### (4S,5R)-4-((triisopropylsilyl)oxy)-5-(((triisopropylsilyl)oxy)methyl)dihydrofuran-2(3H)-one

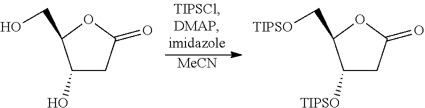

(4S,5R)-4-hydroxy-5-(hydroxymethyl)dihydrofuran-2(3H)-one (151.05 g, 1143 mmol) was dissolved in acetonitrile (1.10 L) with 1H-imidazole (234.00 g, 3437 mmol), and DMAP (6.9790 g, 57.1 mmol). To this suspension was added chlorotriisopropylsilane (587 mL, 2743 mmol) slowly over 45 min. The resulting mixture was stirred at room temperature overnight, then diluted with heptane (1 L), washed with water (1 L), back-extracted with heptane (500 mL), washed with brine, dried (Na₂SO₄), and concentrated to give 550 g of clear oil, which was filtered through silica (1 kg) with 15% MTBE/heptane (12 L), concentrated, and removed residual silanol by azeotroping with toluene (4×200 mL) to give (4S,5R)-4-((triisopropylsilyl)oxy)-5-(((triisopropylsilyl)oxy)methyl)dihydrofuran-2(3H)-one (496.7 g, 1117 mmol, 98% yield) as a colorless oil. ¹H NMR (500 MHz, Chloroform-d) δ 4.69 (dt, J=6.6, 1.8 Hz, 1H), 4.44 (q, J=2.6 Hz, 1H), 3.99-3.81 (m, 2H), 2.91 (dd, J=17.6, 6.6 Hz, 1H), 2.46 (dd, J=17.7, 2.0 Hz, 1H), 1.20-1.00 (m, 42H); MS (ESI (+)) m/z 462 (M+NH₄)⁺.

### EXAMPLE 1B

### (4R,5R)-3-fluoro-4-((triisopropylsilyl)oxy)-5-(((triisopropylsilyl)oxy)methyl)dihydrofuran-2(3H)-one

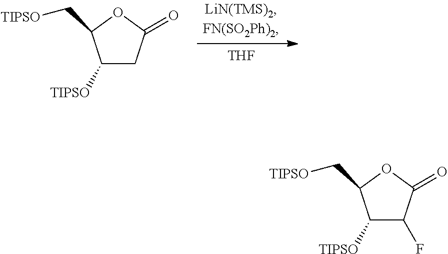

To a solution of (4S,5R)-4-((triisopropylsilyl)oxy)-5-(((triisopropylsilyl)oxy) methyl)dihydrofuran-2(3H)-one (10.08 g, 22.66 mmol) and N-fluoro-N-(phenylsulfonyl)benzenesulfonamide (9.2911 g, 29.5 mmol) in tetrahydrofuran (100 ml) at −78° C. was added a solution of lithium bis(trimethylsilyl)amide (34.5 ml, 1 M in THF, 32.1 mmol) dropwise over 30 min. The resulting solution was stirred for 3 h at −78° C., quenched with 1 M HCl (35 mL), and diluted with heptane (100 mL). The organic layer was washed with water (30 mL), back-extracted the aqueous layer with heptane (20 mL), washed the combined organic layers with saturated aqueous NaHCO₃ (75 mL), and brine (20 mL), dried (Na₂SO₄), and concentrated. The crude product was diluted with heptane (30 mL), filtered through Celite, and concentrated to give an orange oil, which was purified by column chromatography (20-50% toluene/heptane gradient) to give (4R,5R)-3-fluoro-4-((triisopropylsilyl)oxy)-5-(((triisopropylsilyl)oxy)methyl)dihydrofuran-2(3H)-one (7.43 g, 16.05 mmol, 70.8% yield) as a colorless oil. ¹H NMR (500 MHz, Chloroform-d) δ 5.14 (dd, J=51.3, 7.4 Hz, 1H), 4.94 (dt, J=18.8, 7.2 Hz, 1H), 4.19 (dt, J=7.1, 2.3 Hz, 1H), 4.11 (dt, J=12.2, 2.2 Hz, 1H), 3.94 (dd, J=12.1, 2.4 Hz, 1H), 1.20-0.98 (m, 42H); MS (ESI (+)) m/z 463 (M+H)⁺.

### EXAMPLE 1C

### (3S,4R,5R)-3-bromo-3-fluoro-4-((triisopropylsilyl)oxy)-5-(((triisopropylsilyl)oxy)methyl) dihydrofuran-2(3H)-one

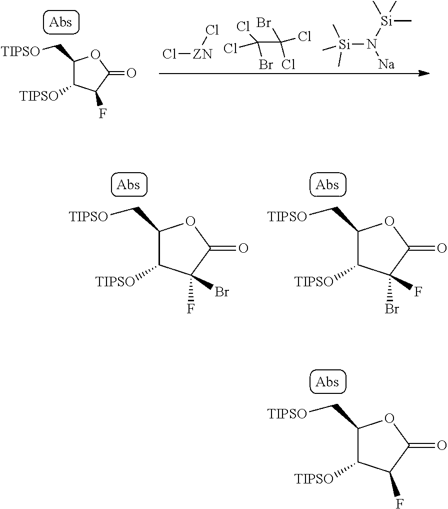

(3 S,4R,5R)-3-fluoro-4-((triisopropylsilyl)oxy)-5-(((triisopropylsilyl)oxy)methyl)dihydrofuran-2(3H)-one (2.52 g, 5.45 mmol) and 1,2-dibromotetrachloroethane (2.128 g, 6.53 mmol) were dissolved in tetrahydrofuran (25 ml) and cooled to an internal temperature of −70° C., added a solution of zinc chloride (5.99 ml, 1 M in Et₂O, 5.99 mmol), allowed to re-cool to −75° C. then added a solution of sodium bis(trimethylsilyl)amide (11.98 ml, 1 M in THF, 11.98 mmol) dropwise over 10 min stirred for 1 h at −78° C., then quenched with MeOH (5 mL), added 1 M HCl (25 mL) and heptane (25 mL), then separated the layers. Washed the organic layer with brine (10 mL), and concentrated to give a colorless oil. The crude product was dissolved in heptane (25 mL), washed with MeCN (3×5 mL), back extracted the combined MeCN layers with heptane (25 mL), washed with MeCN (5 mL), and back extracted with heptane (25 mL). The combined heptane layers were concentrated to give (3S,4R,5R)-3-bromo-3-fluoro-4-((triisopropylsilyl)oxy)-5-(((triisopropylsilyl)oxy)methyl)dihydrofuran-2(3H)-one (2.707 g, 5.00 mmol, 92% yield) as a slightly yellow oil (9:1 d.r. by ¹H NMR). ¹H NMR (400 MHz, Chloroform-d) δ 4.93 (dd, J=4.5, 2.8 Hz, 1H), 4.54-4.40 (m, 1H), 4.14-4.01 (m, 2H), 1.19-1.00 (m, 42H); MS (ESI (+)) m/z 541, 543 (M+H)⁺.

### EXAMPLE 1D

### (3S,4R,5R)-3-bromo-3-fluoro-4-((triisopropylsilyl)oxy)-5-(((triisopropylsilyl)oxy)methyl) tetrahydrofuran-2-ol

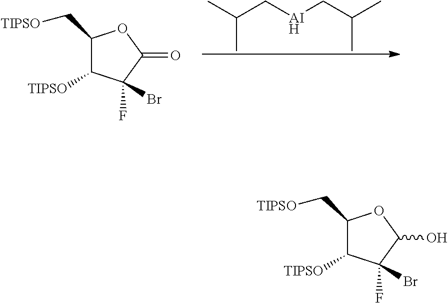

(3S ,4R,5R)-3-bromo-3-fluoro-4-((triisopropylsilyl)oxy)-5-(((triisopropylsilyl)oxy)methyl) dihydrofuran-2(3H)-one (14.52 g, 26.8 mmol) was dissolved in toluene (73 ml) and cooled to an internal temp of −76° C., added a solution of diisobutylaluminum hydride (32.2 ml, 1 M in PhMe, 32.2 mmol) slowly over 10 min. Stirred for 45 min below −75° C., then quenched with MeOH (15 mL), followed by 1 M HCl (150 mL) and EtOAc (150 mL). The resulting mixture was stirred for 40 min at room temp, then the layers were separated. Extracted the aqueous layer with EtOAc (50 mL), then washed the combined organic layers with brine (75 mL), dried over sodium sulfate, filtered and concentrated to give a slightly yellow oil. Which was purified by column chromatography (0-60% CH₂Cl₂/heptane gradient) to give (3S ,4R,5R)-3-bromo-3-fluoro-4-((triisopropylsilyl)oxy)-5-(((triisopropylsilyl)oxy)methyl) tetrahydrofuran-2-ol (12.2 g, 22.44 mmol, 84% yield) as a colorless oil. ¹H NMR showed a 1:1 mixture of anomers. ¹H NMR (400 MHz, Chloroform-d) δ 5.38 (dd, J=12.0, 2.2 Hz, 1H), 5.15 (dd, J=9.6, 5.5 Hz, 1H), 4.86 (dd, J=16.1, 5.7 Hz, 1H), 4.75 (dd, J=6.1, 3.5 Hz, 1H), 4.22 (dtd, J=6.0, 4.2, 1.8 Hz, 1H), 4.08 (dt, J=5.4, 2.4 Hz, 1H), 4.01-3.78 (m, 7H), 3.59 (dd, J=12.0, 1.8 Hz, 1H), 1.22-0.99 (m, 84H); MS (ESI (+)) m/z 525, 527 (M−OH)⁺.

### EXAMPLE 1E

### (2R,3S,4R,5R)-3-bromo-3-fluoro-4-((triisopropylsilyl)oxy)-5-(((triisopropylsilyl)oxy) methyl)tetrahydrofuran-2-yl 4-methoxybenzoate

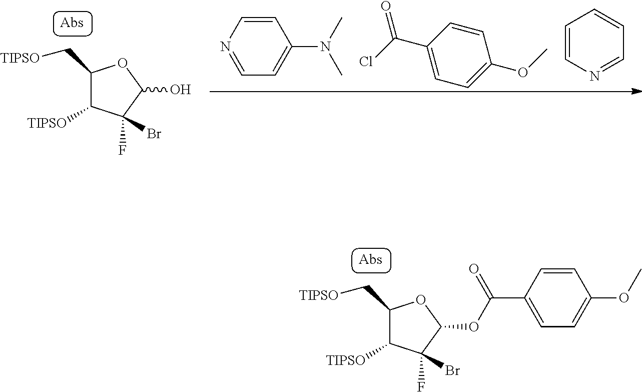

(3S,4R,5R)-3-bromo-3-fluoro-4-((triisopropylsilyl)oxy)-5-(((triisopropylsilyl)oxy)methyl) tetrahydrofuran-2-ol (9.5 g, 17.47 mmol) and DMAP (4-dimethylaminopyridine) (0.213 g, 1.747 mmol) dissolved in Acetonitrile (47.5 ml). Added pyridine (1.7 ml, 21.02 mmol), followed by 4-methoxybenzoyl chloride (2.60 ml, 19.22 mmol) in one portion. The resulting solution, was stirred for 22 h at room temp, then diluted with heptane (100 mL), and 1 M HCl (50 mL). Washed the organic layer with saturated aqueous NaHCO₃ (50 mL), brine (20 mL), and with MeCN (3×20 mL). Back-extracted the combined MeCN layers with heptane (100 mL), and washed with MeCN (2×20 mL). Concentrated the combined heptane layers to give (2R,3S,4R,5R)-3-bromo-3-fluoro-4-((triisopropylsily)oxy)-5-(((triisopropylsilypoxy)methyptetrahydrofuran-2-yl 4-methoxybenzoate (11.456 g, 16.90 mmol, 97% yield) as a colorless oil. ¹H NMR showed a 4:1 mixture of anomers favoring the isomer drawn. ¹H NMR (400 MHz, Chloroform-d) δ 8.08-8.02 (m, 2H), 6.91-6.87 (m, 2H), 6.70 (s, 1H), 4.86 (dd, J=4.2, 2.2 Hz, 1H), 4.30 (dd, J=4.3, 1.0 Hz, 1H), 3.99 (dd, J=6.5, 4.3 Hz, 2H), 3.86 (s, 3H), 1.22-1.02 (m, 42H); MS (ESI (+)) m/z 694, 696 (M+NH₄)⁺.

### EXAMPLE 1F

### 2R,3S,4R,5R)-3-bromo-3-fluoro-4-hydroxy-5-(hydroxymethyl)tetrahydrofuran-2-yl 4-methoxybenzoate

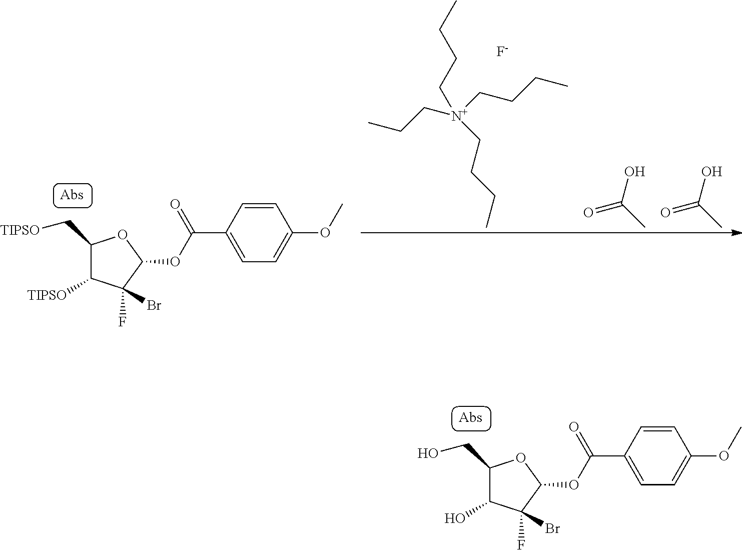

(2R,3S,4R,5R)-3-bromo-3-fluoro-4-((triisopropylsilyl)oxy)-5-(((triisopropylsilyl)oxy)methyl) tetrahydrofuran-2-yl 4-methoxybenzoate (11.456 g, 16.90 mmol) was dissolved in tetrahydrofuran (60 ml) and cooled to 0° C., added acetic acid (0.5 ml, 8.73 mmol), followed by tetra-N-butylammonium fluoride (37.2 ml, 1 M in THF, 37.2 mmol) slowly. Stirred for 1 h at 0° C., then added acetic acid (2.25 ml, 39.3 mmol), concentrated, and chased with EtOAc (20 mL) to give a light bronze oil. Dissolved in EtOAc (50 mL) added 2 M HCl (25 mL), and the mix was stirred vigorously for 5 min, then separated, washed the organic layer with 2 M HCl (2×25 mL), back-extracted the combined aqueous layers with EtOAc (2×25 mL). The combined organic layers were washed with saturated aqueous NaHCO₃ (25 mL), brine (30 mL), dried (Na₂SO₄), and concentrated to give a slightly yellow oil, which was purified by column chromatography (0-35% EtOAc/CH₂Cl₂ gradient) to give (2R,3S,4R,5R)-3-bromo-3-fluoro-4-hydroxy-5-(hydroxymethyl)tetrahydrofuran-2-yl 4-methoxybenzoate (5.74 g, 15.72 mmol, 93% yield) as a colorless gel, 1H NMR showed a roughly 4:1 ratio of anomers favoring the isomer indicated. ¹H NMR (400 MHz, DMSO-d₆) δ 8.01-7.92 (m, 2H), 7.16-7.06 (m, 2H), 6.57 (dd, J=4.5, 0.5 Hz, 1H), 6.49 (d, J=5.8 Hz, 1H), 5.12-5.04 (m, 1H), 4.41 (dt, J=10.2, 6.1 Hz, 1H), 4.17-4.06 (m, 1H), 3.85 (s, 3H), 3.71-3.64 (m, 1H), 3.61-3.54 (m, 1H); MS (ESI (+)) m/z 387, 389 (M+Na)⁺.

### EXAMPLE 1G

### (2R,3S,4R,5R)-3-bromo-3-fluoro-5-(((4-methoxybenzoyl)oxy)methyl)tetrahydrofuran-2,4-diyl bis(4-methoxybenzoate)

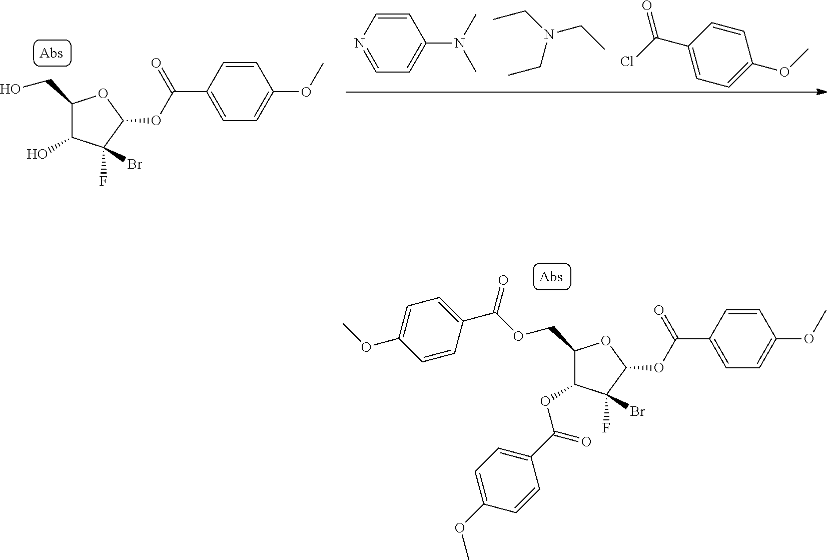

(2R,3S,4R,5R)-3-bromo-3-fluoro-4-hydroxy-5-(hydroxymethyl)tetrahydrofuran-2-yl 4-methoxybenzoate (4.41 g, 12.08 mmol) was dissolved in dichloromethane (40 ml) and cooled to 0° C. Added DMAP (0.148 g, 1.208 mmol) and triethylamine (4.21 ml, 30.2 mmol), followed by 4-methoxybenzoyl chloride (3.60 ml, 26.6 mmol) in one portion. The colorless solution immediately became a suspension, which was stirred for 1 h at 0° C., then quenched with H2O (40 mL). Extracted the aqueous layer with CH₂Cl₂ (20 mL), washed the combined organic layers with 1 M HCl (20 mL), brine (10 mL), and saturated aqueous NaHCO₃ (2×20 mL), dried over sodium sulfate, filtered and concentrated to give a colorless oil. The crude product was dissolved in CH₂Cl₂ (4 mL), then added MeOH (12 mL) dropwise. Stirred for 1 h, then filtered off the resulting white solid and washed with 3:1 MeOH/CH₂Cl₂ (4 mL), and MeOH (8 mL) to give (2R,3S,4R,5R)-3-bromo-3-fluoro-5-(((4-methoxybenzoyl)oxy)methyl)tetrahydrofuran-2,4-diyl bis(4-methoxybenzoate) (5.3793 g, 8.49 mmol, 70.3% yield) as a white solid. ¹H NMR (400 MHz, DMSO-d₆) δ 8.03-7.90 (m, 6H), 7.11-6.95 (m, 6H), 6.74 (s, 1H), 5.87 (d, J=4.3 Hz, 1H), 5.07-4.99 (m, 1H), 4.68 (dd, J=12.4, 3.5 Hz, 1H), 4.60 (dd, J=12.4, 5.3 Hz, 1H), 3.83 (s, 3H), 3.82 (s, 3H), 3.80 (s, 3H); MS (ESI (+)) m/z 650, 652 (M+NH₄)⁺.

### EXAMPLE 1H

### 2,4-Bis((trimethylsilyl)oxy)pyrimidine

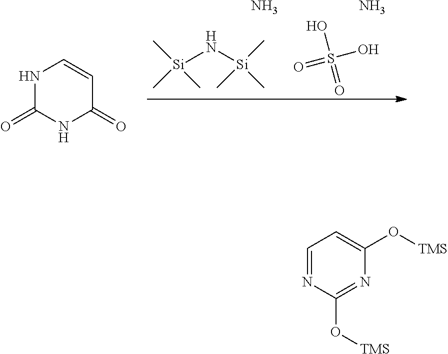

Uracil (100 g, 892 mmol), 1,1,1,3,3,3-hexamethyldisilazane (561 ml, 2676 mmol) and ammonium sulfate (2.358 g, 17.84 mmol) were combined in chlorobenzene (500 ml) and heated to an internal temp of 125° C. for 15 h. The mixture was cooled to <80° C., then concentrated (bath temp 80° C.), backfilling with nitrogen instead of air. The 2,4-bis((trimethylsilyl)oxy)pyrimidine (248.9 g, 971 mmol, 109% yield) thus obtained a slightly yellow oil was used for the next step without purification.

### EXAMPLE 1I

### (2R,3R,4S,5R)-4-Bromo-5-(2,4-dioxo-3,4-dihydropyrimidin-1(2H)-yl)-4-fluoro-2-(((4-methoxybenzoyl)oxy)methyl)tetrahydrofuran-3-yl 4-methoxybenzoate

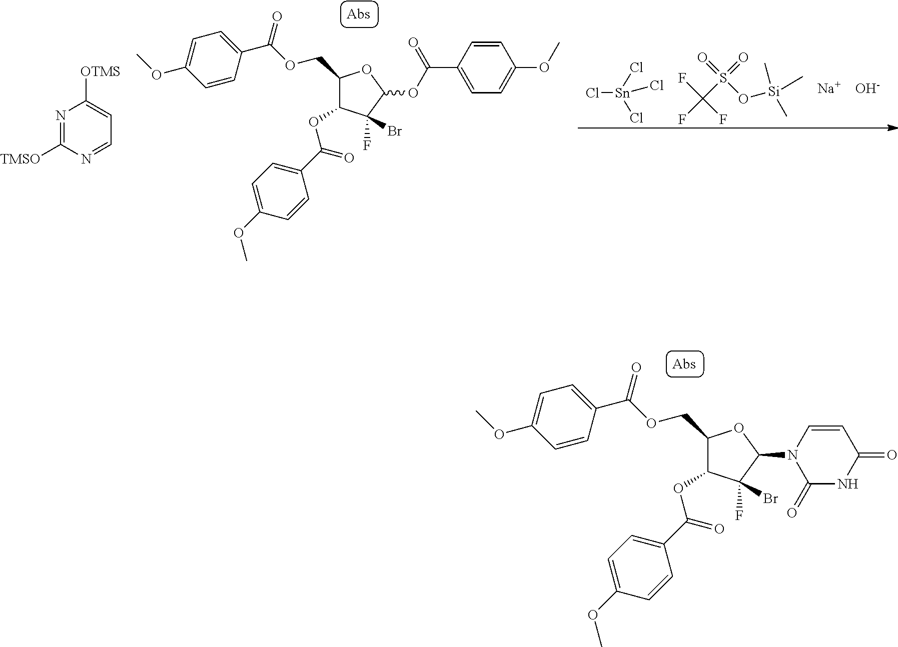

(3S ,4R,5R)-3-Bromo-3-fluoro-5-(((4-methoxybenzoyl)oxy)methyl)tetrahydrofuran-2,4-diyl bis(4-methoxybenzoate) (239 g, 377 mmol) was dissolved in chlorobenzene (5000 ml). 2,4-Bis((trimethylsilyl)oxy)pyrimidine (229 g, 893 mmol) and trimethylsilyl trifluoromethanesulfonate (140 ml, 775 mmol) were added, followed by tin (IV) chloride (180 ml, 1534 mmol). The solution was heated to an internal temperature of 90° C. for 18 h (became gold/orange while heating then darkened overnight).

The reaction mixture was cooled to an internal temp of 25° C., then EtOAc (2.5 L) and Celite (500 g) were added (internal temp <30° C.). The mixture was cooled to 6° C., then 50% w/w sodium hydroxide (400 ml, 7627 mmol) was added slowly over 45 min (internal temp <14° C.). Stirred for 30 min, removed the cooling bath, added additional Celite (500 g), stirred vigorously for 1 h, then filtered through Celite (500 g), washing the solid with EtOAc (3×1.5 L).

The mixture was washed with 0.5 M NaOH (2×1500 mL) and brine (1500 mL). The combined aqueous layers were back extracted with EtOAc (1500 L), and this organic layer was washed with brine (600 mL). The combined organic layers were washed with 1:1 1M HCl brine (1500 mL), then brine (600 mL). The combined organic layers were dried over sodium sulfate and filtered. The filtrate was directly passed through a silica plug (500 g), washing the silica with EtOAc (3 L). The eluent was concentrated to give a tan/orange solid, which was dissolved in chlorobenzene (2.4 L) by heating to 90° C. The solution was slowly cooled to room temp, with seeding at 80° C. with authentic product (1.5 g). After 10 h of mixing, the white slurry was filtered and washed with PhCl (2×240 mL slurry wash). The off-white solid was air-dried on the filter while checking mother liquor and wet cake by HPLC (93.5 pa % at 254 nm for the solid with low loss in the mother liquor.) The free-flowing wet cake solid (175 g) was transferred with PhCl (8 vol, 1.9 L), then heated to 85° C. (solution with a tiny amount of solids still present) followed by cooling to rt over 3 h. The resulting slurry was stirred vigorously at rt for 30 min, filtered, and washed with PhCl (240 mL displacement wash). The white solid was air dried on the filter for 30 min, then in a vacuum oven at 50° C., giving (2R,3R,4S,5R)-4-bromo-5-(2,4-dioxo-3,4-dihydropyrimidin-1(2H)-yl)-4-fluoro-2-(((4-methoxybenzoyl)oxy)methyl)tetrahydrofuran-3-yl 4-methoxybenzoate (66.7 g, 102 mmol, 26.9% potency-adjusted yield). ¹H NMR (400 MHz, DMSO-d₆) δ 11.7 (brs, 1H), 8.04-7.93 (m, 2H), 7.90-7.71 (m, 3H), 7.14-7.05 (m, 2H), 6.99-6.91 (m, 2H), 6.38 (d, J=17.0 Hz, 1H), 6.14 (brs, 1H), 5.73 (d, J=8.1 Hz, 1H), 4.70-4-56 (m, 3H), 3.86 (s, 3H), 3.81 (s, 3H); MS (ESI). m/z 593, 595 [M+H]+.

### EXAMPLE 1J

### 1-((2R,3S,4R,5R)-3-bromo-3-fluoro-4-hydroxy-5-(hydroxymethyl)tetrahydrofuran-2-yl)pyrimidine-2,4(1H,3H)-dione

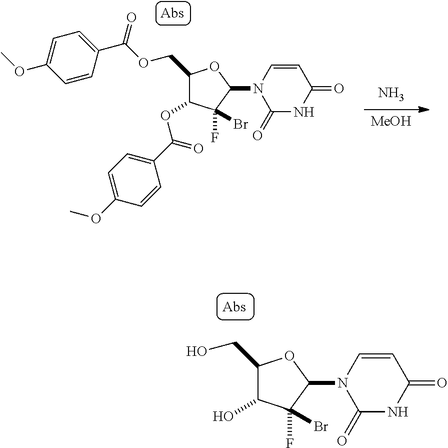

A mixture of (2R,3R,4S,5R)-4-bromo-5-(2,4-dioxo-3,4-dihydropyrimidin-1(2H)-yl)-4-fluoro-2-(((4-methoxybenzoyl)oxy)methyl)tetrahydrofuran-3-yl 4-methoxybenzoate (67.8 g, 103 mmol) in 7 M ammonia/MeOH (369 mL, 2580 mmol) was heated in a sealed Parr shaker at 35° C. for 90 h. The reaction mixture was concentrated, dissolved in water (210 mL) and DCM (420 mL) with warming to 40° C. The layers were separated. The aqueous layer was washed with DCM (2×420 mL) and the DCM layers back-extracted with water (70 mL). The aqueous layer was acidified to pH ˜1 with 12 N HCl (7 mL), extracted with MeTHF (7×210 mL), and concentrated to a light brown solid (40° C. rotovap bath temp). IPAc (210 mL) was added and the mixture concentrated to a light brown solid. Again IPAc (70 mL) was added, then the mixture sonicated and stirred vigorously for 30 min. The resulting white slurry was filtered and washed with minimal IPAc. The white solid was dried in a vacuum oven at 50° C. to constant weight, giving 1-((2R,3S,4R,5R)-3-bromo-3-fluoro-4-hydroxy-5-(hydroxymethyl)tetrahydrofuran-2-yl)pyrimidine-2,4(1H,3H)-dione (27.44 g, 84 mmol, 82% yield). ¹H NMR (400 MHz, DMSO-d₆) δ 11.60 (brs, 1H), 7.84 (d, J=8.2 Hz, 1H), 6.53 (d, J=7.2 Hz, 1H), 6.24 (d, J=16.9 Hz, 1H), 5.75 (dd, J=8.1, 2.1 Hz, 1H), 5.43-5.33 (m, 1H), 4.29 (ddd, J=21.1, 9.2, 7.2 Hz, 1H), 3.90-3.77 (m, 2H), 3.64 (ddd, J=12.6, 5.2, 3.2 Hz, 1H). MS (ESI). m/z 325, 327 [M+H]+.

### EXAMPLE 1K

### Ethyl 2-(((S)-(((2R,3R,4S,5R)-4-bromo-5-(2,4-dioxo-3,4-dihydropyrimidin-1(2H)-yl)-4-fluoro-3-hydroxytetrahydrofuran-2yl)methoxy)(phenoxy)phosphoryl)amino)-2-methylpropanoate

1-((2R,3S,4R,5R)-3-bromo-3-fluoro-4-hydroxy-5-(hydroxymethyl)tetrahydrofuran-2-yl)pyrimidine-2,4(1H,3H)-dione was reacted with

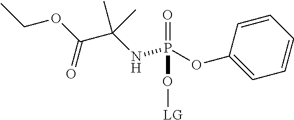

wherein —O-LG is a leaving group. Separated layers, washed the organic layer with 1:1 2N HCl/brine (2×120 mL) and back-extracted the aq layers with 1:1 THF/toluene (54 mL). Washed the organic layers with satd aq NaHCO₃ (120 mL) and brine (60 mL), dried (Na₂SO₄), filtered, and the filtrate was concentrated. The crude material was purified by flash column chromatography (25-80% EtOAc/heptanes gradient elution, then 10-50% ACN/DCM gradient elution). Suitable fractions were combined and concentrated, giving ethyl 2-(((S)-(((2R,3R,4S,5R)-4-bromo-5-(2,4-dioxo-3,4-dihydropyrimidin-1(2H)-yl)-4-fluoro-3-hydroxytetrahydrofuran-2-yl)methoxy)(phenoxy)phosphoryl)amino)-2-methylpropanoate (35.61 g, 59.9 mmol, 71.3% yield). To further purify, 31.7 g was subject to chiral prep SFC purification, giving 27.5 g (87% recovery) clean ethyl 2-(((S)-(((2R,3R,4S,5R)-4-bromo-5-(2,4-dioxo-3,4-dihydropyrimidin-1(2H)-yl)-4-fluoro-3-hydroxy tetrahydrofuran-2-yl)methoxy)(phenoxy)phosphoryl)amino)-2-methylpropanoate. ¹H NMR (501 MHz, DMSO-d₆) δ 11.63 (s, 1H), 7.59 (d, J=8.1 Hz, 1H), 7.43-7.32 (m, 2H), 7.25-7.16 (m, 3H), 6.70 (d, J=6.9 Hz, 1H), 6.25 (d, J=17.6 Hz, 1H), 5.94 (d, J=9.6 Hz, 1H), 5.61 (d, J=8.1 Hz, 1H), 4.43-4.25 (m, 3H), 4.10-4.00 (m, 3H), 1.40 (s, 3H), 1.36 (s, 3H), 1.15 (t, J=7.1 Hz, 3H). MS (ESI). m/z 594, 596 [M+H]+.

Preparative SFC was performed on a THAR/Waters SFC 80 system running under SuperChrom software control. The preparative SFC system was equipped with a CO₂ pump, modifier pump, automated back pressure regulator (ABPR), UV detector, injector, and 6-position fraction collector. The mobile phase comprised of supercritical CO₂ supplied by a bulk tank of 99.5% bone-dry non-certified CO₂ pressurized to 1200 psi with a modifier of MeOH at a flow rate of 70 g/min. UV detection was set to collect at a wavelength of 220 nm, the column compartment was at ambient temperature, and the backpressure regulator was set to maintain 100 bar. The sample was dissolved in MeOH at a concentration of 500 mg/mL and the injection volume was 1 mL. The mobile phase was held isocratically at 20% MeOH:CO₂. The instrument was fitted with a Regis Pirkle-Type Whelk-O (S,S) column with dimensions 21 mm i.d.×250 mm length with 5 μm particles. The di-F impurity was collected at a retention time of 4.71 minutes, the desired 1-((2R,3S,4R,5R)-3-bromo-3-fluoro-4-hydroxy-5-(hydroxymethyl)tetrahydrofuran-2-yl)pyrimidine-2,4(1H,3H)-dione product was collected at a retention time of 5.55 minutes, and the di-prodrug was collected at a retention time of 9.35 minutes.

### EXAMPLE 2

### Ethyl 2-(((S)-(((2R,3R,4R,5R)-4-bromo-4-chloro-5-(2,4-dioxo-3,4-dihydropyrimidin-1(2H)-yl)-3-hydroxytetrahydrofuran-2-yl)methoxy)(phenoxy)phosphoryl)amino)-2-methylpropanoate

### EXAMPLE 2A

### (4S,5R)-4-((tert-butyldimethylsily)oxy)-5-(((tert-butyldimethylsilyl)oxy)methyl) dihydrofuran-2(3H)-one

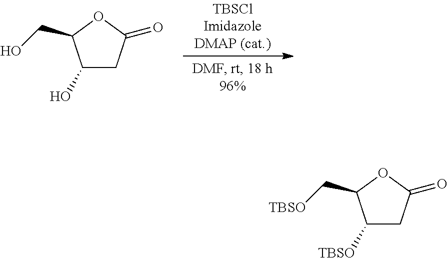 [SMILES: O=C1C[C@H](O)[C@@H](CO)O1 ; CC(C)(C)[Si](C)(C)OC[C@H]1OC(=O)C[C@@H]1O[Si](C)(C)C(C)(C)C]

In a 12 L three-neck round bottom flask under nitrogen, (4S,5R)-4-hydroxy-5-(hydroxymethyl)dihydrofuran-2(3H)-one (366.4 g, 2773 mmol, 1 eq), imidazole (472 g, 6933 mmol, 2.5 eq) and DMF (2.5 L) were charged and stirred at room temperature. At room temperature, TBSCl (878 g, 5824 mmol, 2.1 eq) was added in portions over 10 minutes, due to an exotherm of 15° C. (25° C. to 40° C.). Then DMAP (16.94 g, 139 mmol, 0.05 eq) was added and mixture was allowed to stir at room temperature for 18 hours. Reaction completion was monitored by TLC (Eluent: 15% MTBE/0.5% Et₃N/84.5% heptane). At room temperature, H₂O (5.0 L) was charged to the reaction mixture and allowed to stir for 30 minutes. The solids where filtered through a Buchner Funnel (6.0 L, fine frit). H₂O (0.8 L) was charged to the reaction flask and then agitated and the solids were filtered. H₂O (4.0 L) was charged to the Buchner funnel, the slurry in the Buchner funnel was manually stirred with a spatula and allowed to soak for 10 minutes. Vacuum was reapplied to the Buchner funnel for 30 minutes. The solids were transferred into two trays, which were placed in a vacuum oven at 60° C. with a nitrogen bleed for 18 hours. (4S ,5R)-4-((tert-butyldimethylsilyl)oxy)-5-(((tert-butyldimethylsilyl)oxy)methyl)dihydrofuran-2(3H)-one was isolated as a white crystalline solid (955.3 g, 2649 mmol, 96% yield). ¹H NMR (400 MHz, Chloroform-d) δ 4.50 (ddd, J=6.7, 2.4, 2.4 Hz, 1H), 4.32 (m, 1H), 3.83-3.73 (m, 2H), 2.81 (dd, J=17.6, 6.7 Hz, 1H), 2.38 (dd, J=17.6, 2.6 Hz, 1H), 0.88 (s, 9H), 0.88 (s, 9H), 0.08 (s, 6H), 0.06 (s, 3H), 0.06 (s, 3H).

### EXAMPLE 2B

(4R,5R)-4-((tert-butyldimethylsilyl)oxy)-5-(((tert-butyldimethylsilyl)oxy)methyl)-3-chloro dihydrofuran-2(3H)-one (mixture of diastereomers at C2)

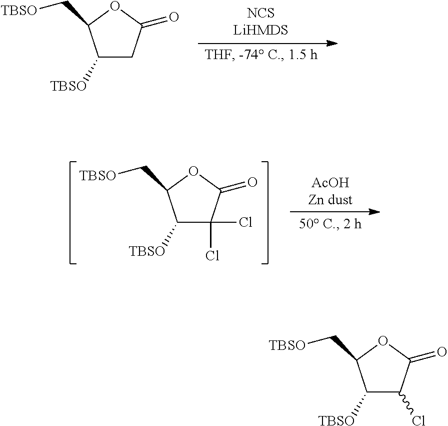

In a 1 L three-neck round bottom flask under nitrogen, (4S,5R)-4-((tert-butyldimethylsilyl)oxy)-5-(((tert-butyldimethylsilyl)oxy)methyl)dihydrofuran-2(3H)-one (14.97 g, 41.6 mmol, 1 eq) and THF (279 mL) were charged and stirred at room temperature and then NCS (12.77 g, 96 mmol, 2.3 eq) was added. The mixture was cooled to −74° C. for 1 hour. LiHMDS (1M solution in THF) (116 ml, 116 mmol, 2.8 eq) was added dropwise over 75 minutes and allowed to stir for 15 minutes. Reaction completion was monitored by TLC (Eluent: 15% MTBE/0.5% Et₃N/84.5%heptane). The reaction was quenched; MeOH (8.4 mL) was added dropwise over 4 minutes at −74° C. The mixture was warmed to −5° C. and Glacial Acetic Acid (25 mL, 10 eq) was charged over 10 minutes. The reaction was warmed to 15° C. and Zinc dust (4.90 g, 74.93 mmol, 1.8 eq) was charged in five portions (5×0.98 g). There was an exotherm of 3-7° C. each time zinc dust was charged. The temperature was allowed to cool back down to 15° C. before charging next portion of Zinc. The mixture was warmed to room temperature and stirred for 1 hour. Reaction completion was monitored by TLC (Eluent: 10% ethyl acetate in heptane). Celite 545 (15 g) was charged to the reaction mixture, which was then filtered over a pad of celite 545 (celite bed was prewashed with heptane). The wet cake was rinsed with heptane (2×300 mL). The filtrate was transferred to a separatory funnel and washed with water (150 mL) and then brine (75 mL). The organic layer was dried over anhydrous Na₂SO₄, filtered, and concentrated under vacuum to afford the crude as a light yellow sludge. Using a disposable filter funnel that was 3 inches in diameter, filled with 60 g of silica gel (70-230 mesh size) to a height of 1.5 inches, the crude sludge was dissolved with heptane (31 mL) and loaded onto the silica plug. The product was eluted with 10% ethyl acetate in heptane (600 mL). The filtrate was concentrated under vacuum and dried under high vacuum overnight. (4R,5R)-4-((tert-butyldimethylsilyl)oxy)-5-(((tert-butyldimethylsilyl)oxy)methyl)-3-chlorodihydrofuran-2(3H)-one (mixture of diastereomers at C2) (15.24g, 38.57mmol, 93% yield) was isolated as a yellow oil with white solids; one diastereomer was a white solid and the other was a yellow oil.

White Solid

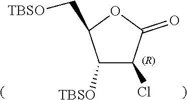

—¹H NMR (600 MHz, Chloroform-d) δ 4.59 (d, J=5.5 Hz, 1H), 4.49 (dd, J=5.5, 2.7 Hz, 1H), 4.38 (m, 1H), 3.92 (dd, J=12.0, 2.7 Hz, 1H), 3.80 (dd, J=12.0, 2.0 Hz, 1H), 0.91 (s, 9H), 0.88 (s, 9H), 0.14 (s, 3H), 0.12 (s, 3H), 0.08 (s, 3H), 0.06 (s, 3H).

Yellow Oil

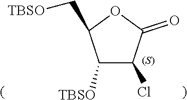

—¹H NMR (400 MHz, CDCl₃) δ 4.61 (m, 1H), 4.41 (d, J=8.0 Hz, 1H), 4.18-4.19 (m, 1H), 3.95-3.99 (m, 1H), 3.78-3.81 (m, 1H), 0.90-0.91 (m, 18H), 0.06-0.19 (m, 12 H).

### EXAMPLE 2C

### (3R,4R,5R)-3-bromo-4-((tert-butyldimethylsilyl)oxy)-5-(((tert-butyldimethylsilyl)oxy)methyl)-3-chlorodihydrofuran-2(3H)-one (mixture of diastereomers at C2)

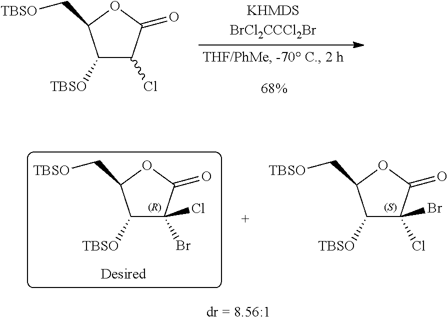

In a 100 mL round bottom flask under nitrogen, (4R,5R)-4-((tert-butyldimethylsilypoxy)-5-(((tert-butyldimethylsilyl)oxy)methyl)-3-chlorodihydrofuran-2(3H)-one (740 mg, 1.873 mmol, 1 eq) (mixture of diastereomers at C2), 1,2-dibromo-1,1,2,2-tetrachloroethane (976 mg, 3.00 mmol, 1.6 eq), and anhydrous THF containing 250 ppm BHT (6.2 mL) were charged to flask. The solution was cooled to −74° C. KHMDS (1.0 M solution in THF) (4.16 mL, 4.16 mmol, 2.2 eq) was added dropwise over 20 minutes. The reaction was then stirred for 2 hours. The reaction was quenched with MeOH (1.38 mL, 18.25 eq) added dropwise over 30 minutes. The reaction was warmed to room temperature. The mixture was concentrated to afford a yellow oil clouded with precipitate. The crude was dissolved with heptane (20 mL), transferred to a separatory funnel and washed with saturated NH₄Cl (8 mL), H₂O (2×10 mL) and brine (1×10 mL). The organic layer was dried over Na₂SO₄, filtered and concentrated. Analyzed by ¹H NMR=dr=8.56:1, in favor of desired diastereomer. The crude material was purified by flash chromatography on an ISCO system (0-40% dichloromethane/heptane gradient). (3R,4R,5R)-3-bromo-4-((tert-butyldimethylsilyl)oxy)-5-(((tert-butyldimethylsilyl)oxy)methyl)-3-chlorodihydrofuran-2(3H)-one (638 mg, 1.27 mmol, dr=8.93:1, 68% yield) was isolated as a yellow oil. ¹H NMR (400 MHz, CDCl₃) δ 4.48 (d, J=7.6 Hz, 1H), 4.11 (d, J=7.6 Hz, 1H), 4.03 (dd, J=12.8, 1.2 Hz, 1H), 3.80 (dd, J=12.8, 0.8 Hz, 1H), 0.97 (s, 9H), 0.89 (s, 9H), 0.26 (s, 3H), 0.18 (s, 3H), 0.09 (s, 3H), 0.09 (s, 3H).

### EXAMPLE 2D

### (4R,5R)-3-bromo-4-((tert-butyldimethylsilyl)oxy)-5-(((tert-butyldimethylsilyl)oxy) methyl)-3-chlorotetrahydrofuran-2-yl benzoate (mixture of diastereomers at C1 & C2)

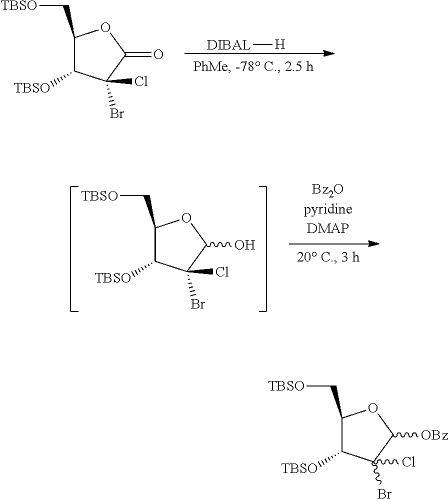

Four reactions were carried out in parallel and combined for workup.

To a solution of (3R,4R,5R)-3-bromo-4-((tert-butyldimethylsilyl)oxy)-5-(((tert-butyldimethylsilyl)oxy)methyl)-3-chlorodihydrofuran-2(3H)-one (200 g, 422 mmol, 1.0 eq) in toluene (1.6 L) was added DIBAL-H (1 M, 464 mL, 1.1 eq) slowly at −78° C. over 30 minutes under N₂. The mixture was stirred at −78° C. for 2 hours to give crude (3R,4R,5R)-3-bromo-4-((tert-butyldimethylsilyl)oxy)-5-(((tert-butyldimethylsilyl)oxy)methyl)-3-chlorotetrahydrofuran-2-ol in a toluene solution. Then pyridine (100 g, 1.27 mol, 3.0 eq), DMAP (77.3 g, 633 mmol, 1.5 eq) and benzoyl benzoate (191 g, 844 mmol, 2.0 eq) were added to the solution sequentially to form a viscous suspension. The mixture was stirred at 20° C. for 3 hours. TLC (Eluent: 10:1 petroleum ether:ethyl acetate, product Rf=0.43) indicated the reaction was complete. The mixture was cooled to −5° C. and quenched with saturated aqueous NH₄Cl (2.24 L) dropwise over 15 minutes. To the mixture was added MTBE (8 L) and saturated aqueous Rochelle's salt (9.6 L). The mixture was warmed to room temperature and stirred for 1 hour. The biphasic mixture was filtered through a frit. The aqueous layer was extracted with MTBE (4.8 L×2). The combined organic layers were washed with 1 N aq. HCl (3.6 L), saturated sodium dicarbonate (3.2 L×3), water (3.2 L) and brine (5.28 L). The organic layer was dried over anhydrous sodium sulfate, filtered and concentrated. The crude product was purified by silica gel plug with 10% ethyl acetate in petroleum ether (24 L) to give (4R,5R)-3-bromo-4-((tert-butyldimethylsilyl)oxy)-5-(((tert-butyldimethylsilyl)oxy)methyl)-3-chlorotetrahydrofuran-2-yl benzoate (mixture of diastereomers) (978 g, 1.69 mol, 99% yield) as a light yellow oil. Major diastereomer: ¹H NMR (400MHz, CDCl₃) δ 8.16-8.18 (m, 1H), 8.04-8.07 (m, 1H), 7.58-7.61 (m, 1H), 7.44-7.48 (m, 2H), 6.52 (d, J=0.6 Hz, 1H), 4.17-4.19 (m, 1H), 4.11 (d, J=6.6 Hz, 1H), 3.89 (dd, J=11.6, 4.2 Hz, 1H), 3.81 (dd, J=11.5, 3.7 Hz, 1H), 0.99 (s, 9H), 0.93 (s, 9H), 0.27 (s, 3H), 0.19 (s, 3H), 0.12 (s, 3H), 0.10 (s, 3H).

### EXAMPLE 2D

### N-(1-((2R,3R,4R,5R)-3-bromo-4-((tert-butyldimethylsilyl)oxy)-5-(((tert-butyl dimethylsilyl)oxy)methyl)-3-chlorotetrahydrofuran-2-yl)-2-oxo-1,2-dihydropyrimidin-4-yl)benzamide

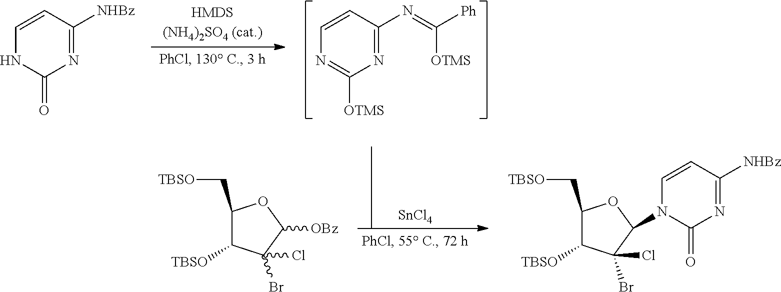

To a solution of N-(2-oxo-1,2-dihydropyrimidin-4-yl)benzamide (176 g, 819 mmol, 2.5 eq) in PhCl (887 mL, 5 volumes) was added (NH₄)₂SO₄ (2.16 g, 16.4 mmol, 0.05 eq), HMDS (265 g, 1.64 mol, 5.0 eq) at 20° C., then the mixture was stirred at 130° C. for 3 hours. The mixture was concentrated under reduced pressure to remove solvent, azeotropic distilled with PhCl (633 mL×2) to give crude (Z)-trimethylsilyl N-(2-((trimethylsilyl)oxy)pyrimidin-4-yl)benzimidate, which was diluted with PhCl (545 mL). To the solution was added (4R,5R)-3-bromo-4-((tert-butyldimethylsilyl)oxy)-5-(((tert-butyldimethylsilyl)oxy)methyl)-3-chlorotetrahydrofuran-2-yl benzoate (mixture of diastereomers) (190 g, 328 mmol, 1.0 eq) in anhydrous PhCl (545 mL, 5 volumes) and SnCl₄ (253 g, 950 mmol, 2.9 eq) at 20° C. The mixture was stirred at 55° C. for 72 hours. TLC (Eluent: dichloromethane:methanol=20:1, Rf=0.20) showed the reaction was complete. The mixture was cooled to 0° C. and quenched with aqueous potassium carbonate solution (3.8 L). The mixture was stirred at 23° C. for 30 minutes, diluted with DCM (10 L) and filtered. The filter cake was washed with DCM (4 L×3), and the aqueous layer was extracted with DCM (3 L). The combined organic layers were washed with brine (3 L), dried over sodium sulfate, filtered, and concentrated to afford the crude product. The crude product was dissolved in DCM (1.2 L), and heptane (2.4 L) was added. The mixture was stirred at 23° C. for 12 hours, and filtered. The filter cake was washed with 10% dichloromethane in heptane (1.2 L×2) and dried to give N-(1-((2R,3R,4R,5R)-3-bromo-4-((tert-butyldimethylsilyl)oxy)-5-(((tert-butyldimethylsilyl)oxy)methyl)-3-chlorotetrahydrofuran-2-yl)-2-oxo-1,2-dihydropyrimidin-4-yl)benzamide (190 g, 141 mmol, 43% yield) as a white solid. ¹H NMR (400MHz, CDCl₃) 6 8.72 (br. s, 1H), 8.35-8.36 (m, 1H), 7.91-7.92 (m, 2H), 7.61-7.64 (m, 1H), 7.51-7.55 (m, 3H), 7.00 (s, 1H), 4.22 (d, J=8.4 Hz, 1H), 4.09-4.12 (m, 1H), 3.99 (d, J=8.4 Hz, 1H), 3.83-3.87 (m, 1H), 1.00 (s, 9H), 0.98 (s, 9H), 0.25 (s, 3H), 0.17 (s, 3H), 0.17 (s, 6H). MS m/z calc. for C₂₈H₄₃N₃O₅Si₂ClBr [M+H]:674.1; found: 674.0.

### EXAMPLE 2E

### 1-((2R,3R,4R,5R)-3-bromo-3-chloro-4-hydroxy-5-(hydroxymethyl)tetrahydrofuran-2-yl)pyrimidine-2,4(1H,3H)-dione

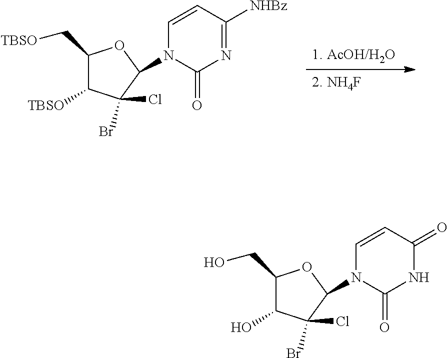

To a 5 L three-neck round-bottom flask, N-(1-((2R,3R,4R,5R)-3-bromo-4-((tert-butyldimethylsilyl)oxy)-5-(((tert-butyldimethylsilyl)oxy)methyl)-3-chlorotetrahydrofuran-2-yl)-2-oxo-1,2-dihydropyrimidin-4-yl)benzamide (290 g, 409 mmol, 1 eq), acetic acid (1.98 L), and water (496 mL) were added resulting in slurry. The mixture was stirred at 100° C. for 4 hours. The reaction mixture was cooled to 23° C. and concentrated to a tan solid, which was azeotroped 2 times with 300 mL methanol. Methanol (1.50 L) and ammonium fluoride (76 g, 2.05 mol) were added to the mixture, which was then stirred at 62° C. for 18 hours. The solution was concentrated to 150 mL and then diluted with 3 L ethyl acetate. The slurry was filtered through a frit containing 1.2 kg Celite 545 and eluted with 38 L ethyl acetate and then 10 L of 10% methanol in ethyl acetate. The solution was concentrated and the mixture was re-dissolved in 0.5 L ethyl acetate. The resulting solution was passed through 0.70 kg of Celite 545 and eluted with 15 L ethyl acetate. The combined filtrates were concentrated and subjected to ISCO purification (5 kg, 0-10% isopropanol in dichloromethane gradient), to obtain 1-((2R,3R,4R,5R)-3-bromo-3-chloro-4-hydroxy-5-(hydroxymethyl)tetrahydrofuran-2-yl)pyrimidine-2,4(1H,3H)-dione (115.9 g, 77.7% yield) as a white solid. ¹H NMR (400 MHz, DMSO-d6) δ 11.55 (s, 1H), 8.09 (d, J=8.2 Hz, 1H), 6.83 (d, J=5.9 Hz, 1H), 6.62 (s, 1H), 5.71 (d, J=8.2, 1H), 5.49 (m, 1H), 4.03 (dd, J=8.8, 5.8 Hz, 1H), 3.87-3.78 (m, 2H), 3.65 (m, 1H). MS m/z calc. for C₉H₁₀BrClN₂O₅ [M+H]:340.95, found 341.05.

### EXAMPLE 2F

### ethyl 2-(((S)-(((2R,3R,4R,5R)-4-bromo-4-chloro-5-(2,4-dioxo-3,4-dihydropyrimidin-1(2H)-yl)-3-hydroxytetrahydrofuran-2-yl)methoxy)(phenoxy)phosphoryl)amino)-2-methylpropanoate

1-((2R,3R,4R,5R)-3-bromo-3-chloro-4-hydroxy-5-(hydroxymethyl)tetrahydrofuran-2-yl)pyrimidine-2,4(1H,3H)-dione was reacted with

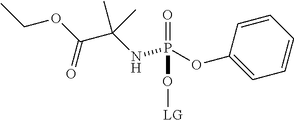

wherein —O-LG is a leaving group. The organic and aqueous layers were separated. The organic layer was extracted with 2×175 mL 1M HCl. The aqueous layer was then back-extracted with 350 mL ethyl acetate. The combined organic layers were washed with brine, dried over sodium sulfate, and concentrated. Two batches of crude product were combined and subjected to ISCO purification (3 kg, 50-100% ethyl acetate in heptane gradient), to obtain ethyl 2-(((S)-(((2R,3R,4R,5R)-4-bromo-4-chloro-5-(2,4-dioxo-3,4-dihydropyrimidin-1(2H)-yl)-3-hydroxytetrahydrofuran-2-yl)methoxy)(phenoxy)phosphoryl)amino)-2-methylpropanoate (56 g, 50% yield) as a white solid. ¹H NMR (400 MHz, DMSO-d6) δ 11.60 (s, 1H), 7.68 (d, J=8.2 Hz, 1H), 7.44-7.34 (m, 2H), 7.27-7.15 (m, 3H), 7.03 (d, J=5.7 Hz, 1H), 6.64 (s, 1H), 5.98 (d, J=9.6 Hz, 1H), 5.54 (d, J=8.1 Hz, 1H), 4.43-4.27 (m, 2H), 4.16-4.08 (m, 1H), 4.03 (q, J=7.1 Hz, 2H), 1.39 (s, 3H), 1.35 (s, 3H), 1.14 (t, J=7.1 Hz, 3H). MS m/z calc. for C₂₁H₂₆BrClN₃O₉P [M+]:609.0, found 609.7.

### EXAMPLE 3-1

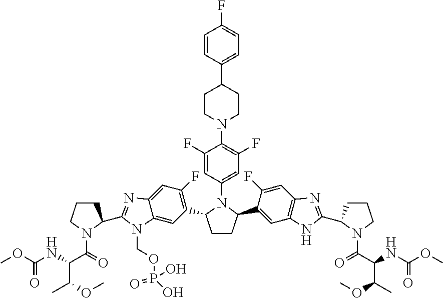

To a solution of dimethyl ((2S,2′S,3R,3′R)-((2S,2′S)-2,2′-(6,6′-((2R,5R)-1-(3,5-difluoro-4-(4-(4-fluorophenyl)piperidin-1-yl)phenyl)pyrrolidine -2,5-diyl)bis (5-fluoro-1H-benzo [d]imidazole -6,2-diyl))bis(pyrrolidine-2,1-diyl))bis(3-methoxy-1-oxobutane-2,1-diyl))dicarbamate (1.0 g, 0.90 mmol) in anhydrous N,N-dimethylformamide (10.0 mL) at 0° C. under a dry N₂ atmosphere was added potassium hydride (108 mg, 2.7 mmol), and the resulting mixture was stirred at 0° C. for 30 min before di-tert-butyl (chloromethyl)phosphate (0.93 g, 3.6 mmol) was added. The resulting mixture was allowed to warm to room temperature and stirred for 2 days. The mixture was partitioned between 0.5N aqueous HCl and a 3:1 mixture of dichloromethane:isopropanol (3×). The combined organic layers were dried over sodium sulfate, filtered and concentrated in vacuo. The residue was dissolved in 1,4-dioxane (4 mL). To the solution was added 4N HCl in dioxane (2 mL), resulting in a precipitate. The mixture was concentrated by evaporation of solvents, and the residue was purified on C18 HPLC using a solvent gradient of 5-35% acetonitrile in water containing 0.1% TFA. Fractions containing pure products were combined and concentrated by lyophilization. The title compound was the first of 2 products to elute during the HPLC purification step, and was obtained as a colorless solid (59 mg); MS (ESI) m/z 1223.3 [M+H]⁺.

### EXAMPLE 3-2

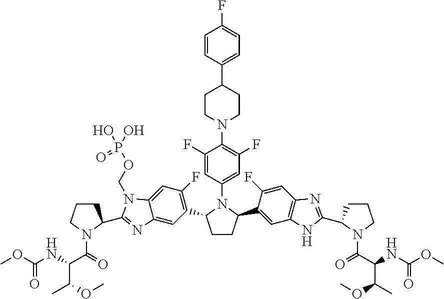

The title compound was prepared using the method described for Example 3-1. It was the second of 2 products to elute during the HPLC purification step, and was obtained as a colorless solid (85 mg); MS (ESI) m/z 1223.2 [M+H]⁺.

### EXAMPLE 3-3

### Dimethyl {[(2R,5R)-1-{3,5-difluoro-4-[4-(4-fluorophenyl)piperidin-1-yl]phenyl}pyrrolidine-2,5-diyl]bis({5-fluoro-1-[(phosphonooxy)methyl]-1H-benzimidazole-6,2-diyl}(2S)pyrrolidine-2,1-diyl [(2S,3R)-3-methoxy-1-oxobutane-1,2-diyl]) }biscarbamate

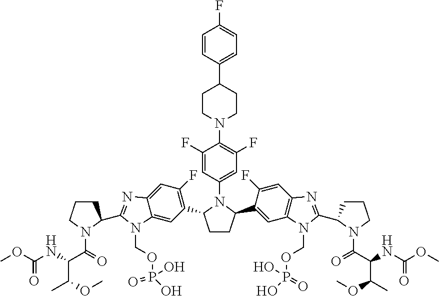

To a solution of dimethyl ((2S,2′S,3R,3′R)-((2S,2′S)-2,2′-(5,5′-((2R,5R)-1-(3,5-difluoro-4-(4-(4-fluorophenyl)piperidin-1-yl)phenyl)pyrrolidine-2,5-diyl)bis (6-fluoro-1H-benzo [d]imidazole -5,2-diyl))bis(pyrrolidine-2,1-diyl))bis(3-methoxy-1-oxobutane-2,1-diyl))dicarbamate (0.111 g, 0.1 mmol) in dimethyl sulfoxide (2.5 mL) was added sodium hydride (60% suspension in mineral oil, 0.020 g, 0.500 mmol), and the resulting mixture was stirred at room temperature for 20 min to give a clear solution. This solution was added drop-wise to a solution of di-tert-butyl (chloromethyl)phosphate (0.129 g, 0.500 mmol) in dimethyl sulfoxide (1.3 mL). The resulting mixture was stirred at room temperature for ca. 16 hr, and the reaction was quenched by adding a drop of water. The mixture was purified by C18HPLC using a solvent gradient of 5-100% acetonitrile in water (0.1% TFA) to give a mixture of 3 bis-phosphomethyl products. This mixture was dissolved in dichloromethane (2.5 mL), trifluoroacetic acid (0.4 mL) was added, and the resulting mixture was stirred at room temperature for 20 min. The reaction mixture was concentrated and the products were separated on C18 HPLC using a solvent gradient of 5-35% acetonitrile in water (0.1% TFA). Fractions containing the pure products were combined and concentrated by lyophilization. The title compound was the first of 3 products to elute, and was obtained as a colorless solid (6 mg); MS (ESI) m/z 1333 [M+H]⁺.

### EXAMPLE 3-4

### Methyl {(2S,3R)-1-[(2S)-2-{5-[(2R,5R)-1-{3,5-difluoro-4-[4-(4-fluorophenyl)piperidin-1-yl]phenyl}-5-{5-fluoro-2-[(2S)-1-{(2S,3R)-3-methoxy-2-[(methoxycarbonyl)amino]butanoyl}pyrrolidin-2-yl]-1-[(phosphonooxy)methyl]-1H-benzimidazol-6-yl}pyrrolidin-2-yl]-6-fluoro-1-[(phosphonooxy)methyl]-1H-benzimidazol-2-yl}pyrrolidin-1-yl]-3-methoxy-1-oxobutan-2-yl}carbamate

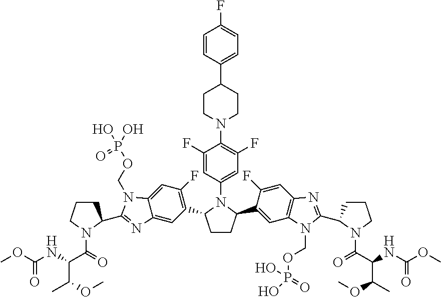

The title compound was prepared using the procedure described for Example 3-3. It was the second of 3 products to elute during the final HPLC purification step, and was obtained as a colorless solid (18 mg). MS (ESI) m/z 1333 [M+H]⁺.

### EXAMPLE 3-5

### Dimethyl {[(2R,5R)-1-{3,5-difluoro-4-[4-(4-fluorophenyl)piperidin-1-yl]phenyl}pyrrolidine-2,5-diyl]bis({6-fluoro-1-[(phosphonooxy)methyl]-1H-benzimidazole-5,2-diyl}(2S)pyrrolidine-2,1-diyl[(2S,3R)-3-methoxy-1-oxobutane -1,2-diyl])}biscarbamate

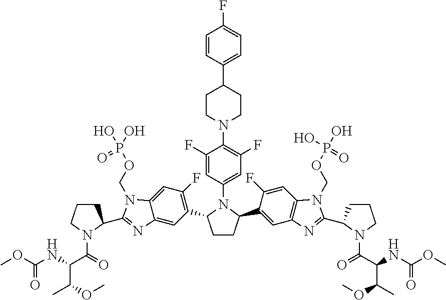

The title compound was prepared using the procedure described for Example 3-3. It was the third of 3 products to elute during the final HPLC purification step, and was obtained as a colorless solid (8 mg). MS (ESI) m/z 1333 [M+H]⁺.

### EXAMPLE 3-6

### (2S,2′S)—((S,S,R)-5,5′-((2R,5R)-1-(3,5-difluoro-4-(4-(4-fluorophenyl)piperidin-1-yl)phenyl)pyrrolidine-2,5-diyl)bis(6-fluoro-2-((S)-1-((2S,3R)-3-methoxy-2-((methoxycarbonyl)amino)butanoyl)pyrrolidin-2-yl)-1H-benzo[d]imidazole-5,1-diyl))bis(methylene) bis(2-amino-3-methylbutanoate)

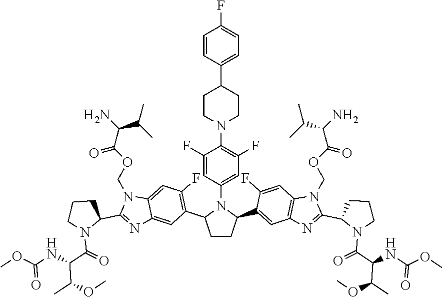

### EXAMPLE 3-6A

### (2S,2′S)—((S,S,R)-5,5′-((2R,5R)-1-(3,5-difluoro-4-(4-(4-fluorophenyl)piperidin-1-yl)phenyl)pyrrolidine-2,5-diyl)bis(6-fluoro-2-((S)-1-((2S,3R)-3-methoxy-2-((methoxycarbonyl)amino)butanoyl)pyrrolidin-2-yl)-1H-benzo[d]imidazole-5,1-diyl))bis(methylene) bis(2-((tert-butoxycarbonyl)amino)-3-methylbutanoate)

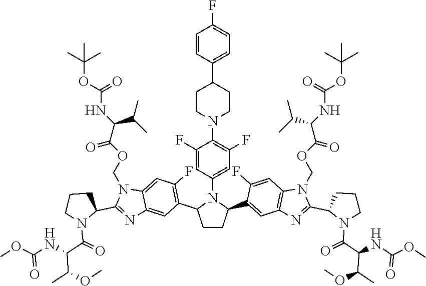

To a solution of dimethyl ((2S,2′S,3R,3′R)-((2S,2′S)-2,2′-(6,6′-((2R,5R)-1-(3,5-difluoro-4-(4-(4-fluorophenyl)piperidin-1-yl)phenyl)pyrrolidine-2,5-diyl)bis(5-fluoro-1H-benzo[d]imidazole-6,2-diyl))bis(pyrrolidine-2,1-diyl))bis(3-methoxy-1-oxobutane-2,1-diyl))dicarbamate (0.50 g, 0.45 mmol) in anhydrous N,N-dimethylformamide (5.0 mL) at 0° C. under a dry N₂ atmosphere was added cesium carbonate (0.512 g, 1.57 mmol), followed by (S)-chloromethyl 2-((tert-butoxycarbonyl)amino)-3-methylbutanoate (0.36 g, 1.35 mmol). The resulting mixture was allowed to slowly warm to room temperature and was stirred for 3 hr. The mixture was partitioned between 0.5N aqueous HCl and ethyl acetate (3×). The combined organic layers were dried over sodium sulfate, filtered and concentrated in vacuo. The crude product mixture was separated by column chromatography on silica gel using a solvent gradient of 0-10% methanol in dichloromethane. The title compound was the first major product to elute, and was obtained as a colorless solid (109 mg).

### EXAMPLE 3-6B

### (2S,2′S)—((S,S,R)-5,5′-((2R,5R)-1-(3,5-difluoro-4-(4-(4-fluorophenyl)piperidin-1-yl)phenyl)pyrrolidine-2,5-diyl)bis(6-fluoro-2-((S)-1-((2S,3R)-3-methoxy-2-((methoxycarbonyl)amino)butanoyl)pyrrolidin-2-yl)-1H-benzo[d]imidazole-5,1-diyl))bis(methylene) bis(2-amino-3-methylbutanoate)

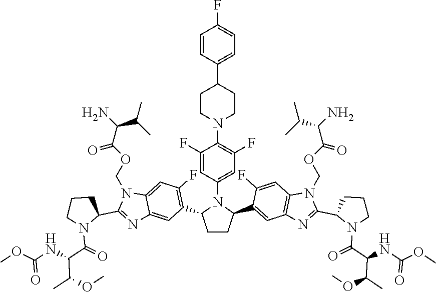

To a solution of the product from Example 3-6A (50 mg, 0.032 mmol) in dichloromethane (4 mL) was added trifluoroacetic acid (1 mL), and the resulting solution was stirred at room temperature for 90 min. The solution was concentrated in vacuo, and the crude product was purified by column chromatography on silica gel using a solvent gradient of 0-10% methanol (containing 2% by volume of concentrated ammonium hydroxide) in dichloromethane. The title compound was obtained as a colorless solid (27 mg); MS (ESI) m/z 1371.3 [M+H]⁺.

### EXAMPLE 3-7

### (S)-(5-((2R,5R)-5-(1-((((S)-2-amino-3-methylbutanoyl)oxy)methyl)-5-fluoro-2-((S)-1-((2S,3R)-3-methoxy-2-((methoxycarbonyl)amino)butanoyl)pyrrolidin-2-yl)-1H-benzo[d]imidazol-6-yl)-1-(3,5-difluoro-4-(4-(4-fluorophenyl)piperidin-1-yl)phenyl)pyrrolidin-2-yl)-6-fluoro-2-((S)-1-((2S,3R)-3-methoxy-2-((methoxycarbonyl)amino)butanoyl)pyrrolidin-2-yl)-1H-benzo[d]imidazol-1-yl)methyl 2-amino-3-methylbutanoate

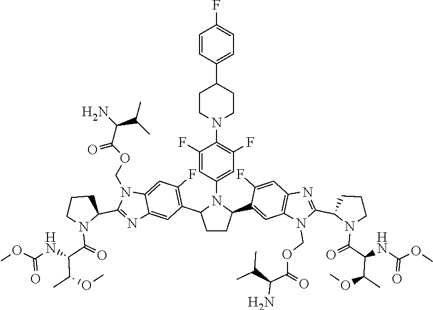

### EXAMPLE 3-7A

### (S)-(5-((2R,5R)-5-(1-((((S)-2-((tert-butoxycarbonyl)amino)-3-methylbutanoyl)oxy)methyl)-5-fluoro-2-((S)-1-((2S,3R)-3-methoxy-2-((methoxycarbonyl)amino)butanoyl)pyrrolidin-2-yl)-1H-benzo[d]imidazol-6-yl)-1-(3,5-difluoro-4-(4-(4-fluorophenyl)piperidin-1-yl)phenyl)pyrrolidin-2-yl)-6-fluoro-2-((S)-1-((2S,3R)-3-methoxy-2-((methoxycarbonyl)amino)butanoyl)pyrrolidin-2-yl)-1H-benzo[d]imidazol-1-yl)methyl 2-((tert-butoxycarbonyl)amino)-3-methylbutanoate

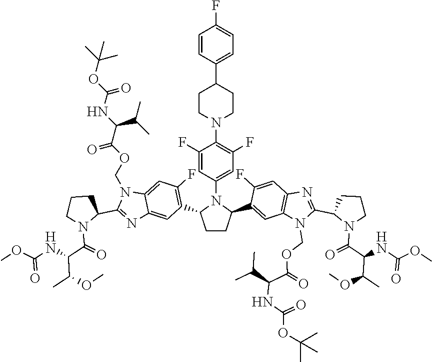

The title compound was prepared using the method described in Example 3-6A. It was the second product to elute during the chromatography step, and was isolated as a colorless solid (383 mg). This product is a mixture of Example 3-7A and Example 3-8A.

### EXAMPLE 3-7B

### (S)-(5-((2R,5R)-5-(1-((((S)-2-amino-3-methylbutanoyl)oxy)methyl)-5-fluoro-2-((S)-1-((2S,3R)-3-methoxy-2-((methoxycarbonyl)amino)butanoyl)pyrrolidin-2-yl)-1H-benzo[d]imidazol-6-yl)-1-(3,5-difluoro-4-(4-(4-fluorophenyl)piperidin-1-yl)phenyl)pyrrolidin-2-yl)-6-fluoro-2-((S)-1-((2S,3R)-3-methoxy-2-((methoxycarbonyl)amino)butanoyl)pyrrolidin-2-yl)-1H-benzo[d]imidazol-1-yl)methyl 2-amino-3-methylbutanoate

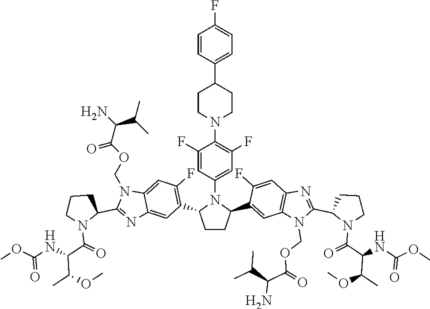

To a solution of the product from Example 3-7A (0.38 g) in dichloromethane (4 mL) was added trifluoroacetic acid (1 mL), and the resulting solution was stirred at room temperature for 90 min. The solution was concentrated in vacuo, and the crude product was purified by column chromatography on silica gel using a solvent gradient of 0-10% methanol (containing 2% by volume of concentrated ammonium hydroxide) in dichloromethane. The title compound was the second of 2 products to elute, and was obtained as a colorless solid (119 mg); MS (ESI) m/z 1371.3 [M+H]⁺.

### EXAMPLE 3-8

### (S)-(5-((2R,5R)-1-(3,5-difluoro-4-(4-(4-fluorophenyl)piperidin-1-yl)phenyl)-5-(5-fluoro-2-((S)-1-((2S ,3R)-3-methoxy-2-((methoxycarbonyl)amino)butanoyl)pyrrolidin-2-yl)-1H-benzo[d]imidazol-6-yl)pyrrolidin-2-yl)-6-fluoro-2-((S)-1-((2S,3R)-3-methoxy -2-((methoxycarbonyl)amino)butanoyl)pyrrolidin-2-yl)-1H-benzo[d]imidazol-1-yl)methyl 2-amino-3-methylbutanoate

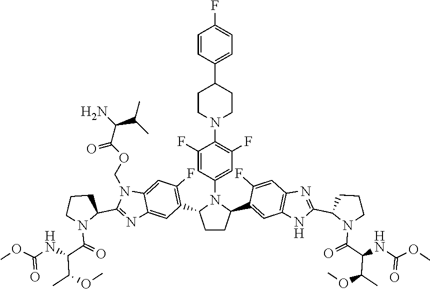

### EXAMPLE 3-8A

### (S)-(5-((2R,5R)-1-(3,5-difluoro-4-(4-(4-fluorophenyl)piperidin-1-yl)phenyl)-5-(5-fluoro-2-((S)-1-((2S,3R)-3-methoxy-2-((methoxycarbonyl)amino)butanoyl)pyrrolidin-2-yl)-1H-benzo[d]imidazol-6-yl)pyrrolidin-2-yl)-6-fluoro-2-((S)-1-((2S,3R)-3-methoxy-2-((methoxycarbonyl)amino)butanoyl)pyrrolidin-2-yl)-1H-benzo[d]imidazol-1-yl)methyl 2-((tert-butoxycarbonyl)amino)-3-methylbutanoate

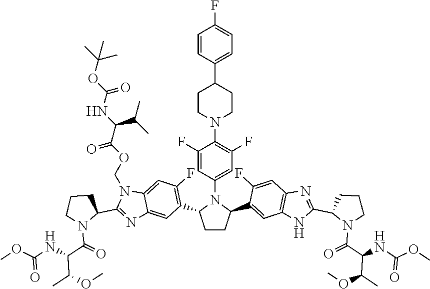

The title compound was prepared using the method described in Example 3-6A. It was the second product to elute during the chromatography step, and was isolated as a colorless solid (383 mg). This product is a mixture of Example 3-8A and Example 3-7A.

### EXAMPLE 3-8B

### (S)-(5-((2R,5R)-1-(3,5-difluoro-4-(4-(4-fluorophenyl)piperidin-1-yl)phenyl)-5-(5-fluoro-2-((S)-1-((2S,3R)-3-methoxy-2-((methoxycarbonyl)amino)butanoyl)pyrrolidin-2-yl)-1H-benzo[d]imidazol-6-yl)pyrrolidin-2-yl)-6-fluoro-2-((S)-1-((2S,3R)-3-methoxy-2-((methoxycarbonyl)amino)butanoyl)pyrrolidin-2-yl)-1H-benzo[d]imidazol-1-yl)methyl 2-amino-3-methylbutanoate

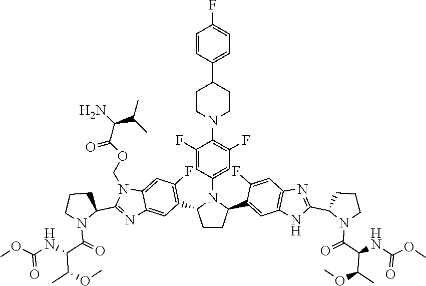

The title compound was isolated from the chromatography step in Example 3-7B. It was the first of 2 products to elute, and was obtained as a colorless solid (108 mg); MS (ESI) m/z 1242.4 [M+H]⁺.

### EXAMPLE 3-9

### ((S,S,R)-5,5′-((2R,5R)-1-(3,5-difluoro-4-(4-(4-fluorophenyl)piperidin-1-yl)phenyl)pyrrolidine-2,5-diyl)bis(6-fluoro-2-((S)-1-((2S,3R)-3-methoxy-2-((methoxycarbonyl)amino)butanoyl)pyrrolidin-2-yl)-1H-benzo[d]imidazole-5,1-diyl))bis(methylene) bis(2,2-dimethylpropanoate)

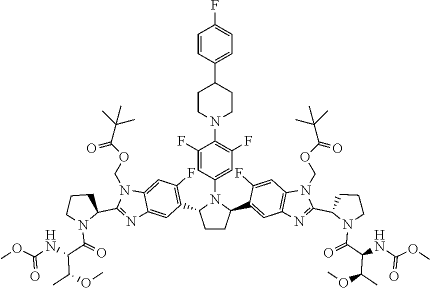

To a solution of dimethyl ((2S,2′S,3R,3′R)-((2S,2′S)-2,2′-(6,6′-((2R,5R)-1-(3,5-difluoro-4-(4-(4-fluorophenyl)piperidin-1-yl)phenyl)pyrrolidine-2,5-diyl)bis(5-fluoro-1H-benzo[d]imidazole-6,2-diyl))bis(pyrrolidine-2,1-diyl))bis(3-methoxy-1-oxobutane-2,1-diyl))dicarbamate (0.10 g, 0.09 mmol) in anhydrous N,N-dimethylformamide (1.0 mL) under a dry N₂ atmosphere was added cesium carbonate (0.059 g, 0.18 mmol), followed by chloromethyl pivalate (0.039 mL, 0.27 mmol). The resulting mixture was stirred for 3 hr. The mixture was partitioned between 0.5N aqueous HCl and ethyl acetate (3×). The combined organic layers were dried over sodium sulfate, filtered and concentrated in vacuo. The crude product mixture was separated by column chromatography on silica gel using a solvent gradient of 0-5% methanol in dichloromethane. The title compound was the first major product to elute, and was obtained as a colorless solid (33 mg); MS (ESI) m/z 1341.5 [M+H]⁺.

### EXAMPLE 3-10

### (5-((2R,5R)-1-(3,5-difluoro-4-(4-(4-fluorophenyl)piperidin-1-yl)phenyl)-5-(5-fluoro-2-((S)-1-((2S,3R)-3-methoxy-2-((methoxycarbonyl)amino)butanoyl)pyrrolidin-2-yl)-1-((pivaloyloxy)methyl)-1H-benzo[d]imidazol-6-yl)pyrrolidin-2-yl)-6-fluoro-2-((S)-1-((2S,3R)-3-methoxy-2-((methoxycarbonyl)amino)butanoyl)pyrrolidin-2-yl)-1H-benzo[d]imidazol-1-yl)methyl pivalate

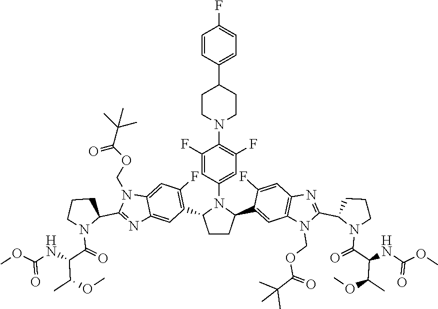

The title compound was prepared using the method described in Example 3-9. It was the second of two major products to elute during the chromatography step, and was obtained as a colorless solid (18 mg); MS (ESI) m/z 1341.5 [M+H]⁺.

The foregoing description of the present invention provides illustration and description, but is not intended to be exhaustive or to limit the invention to the precise one disclosed. Modifications and variations are possible in light of the above teachings or may be acquired from practice of the invention. Thus, it is noted that the scope of the invention is defined by the claims and their equivalents.

What is claimed is:

1. A pharmaceutical composition comprising (1) 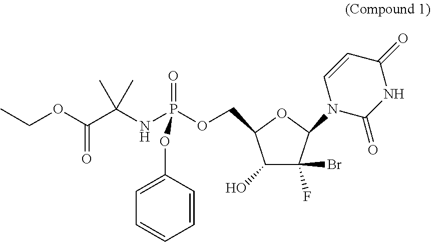 [SMILES: CCOC(=O)C(C)(C)N[P@](=O)(OC[C@H]1O[C@@H](n2ccc(=O)[nH]c2=O)[C@@](F)(Br)[C@@H]1O)Oc1ccccc1]
or a pharmaceutically acceptable salt thereof, and (2) 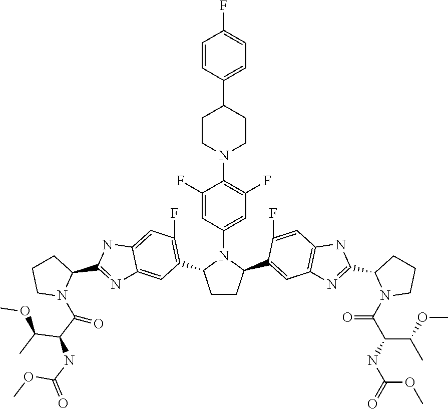
or a pharmaceutically acceptable salt thereof, wherein said pharmaceutical composition is an orally administered dosage form.

2. The pharmaceutical composition of claim 1, which is a tablet.

3. A pharmaceutical composition comprising (1) Compound 1 or a pharmaceutically acceptable salt thereof and (2) a prodrug of Compound 2a (such as one of Compounds 2b-2k or Examples 3-1 to 3-10) or a pharmaceutically acceptable salt of said prodrug, wherein said pharmaceutical composition is an orally administered dosage form.

4. The pharmaceutical composition of claim 3, which is a tablet.

5. A pharmaceutical composition comprising (1) Compound 1 or a pharmaceutically acceptable salt thereof, and (2) 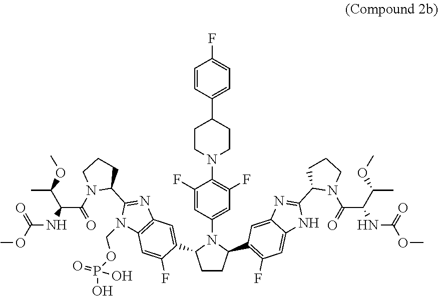
or a pharmaceutically acceptable salt thereof, wherein said pharmaceutical composition is an orally administered dosage form.

6. The pharmaceutical composition of claim 5, which is a tablet.

7. A pharmaceutical composition comprising (1) Compound 1 or a pharmaceutically acceptable salt thereof, and (2) 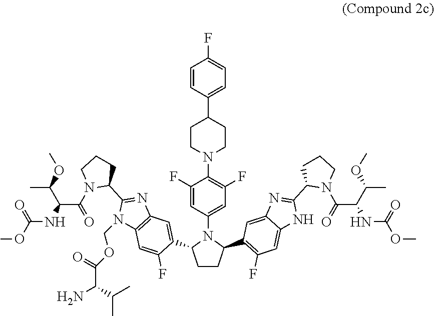
or a pharmaceutically acceptable salt thereof, wherein said pharmaceutical composition is an orally administered dosage form.

8. The pharmaceutical composition of claim 7, which is a tablet.

9. A method of treating HCV infection, comprising administering a pharmaceutical composition according to one of claims 1-8, 23-27 and 33 to a patient in need thereof.

10. The method of claim 9, wherein said a pharmaceutical composition is administered to said patient once daily, and said treatment does not include administration of either interferon or ribavirin to said patient, and wherein said treatment lasts for 4, 5, 6, 7, 8, 9, 10, 11 or 12 weeks.

11. The method of claim 10, wherein said treatment lasts for 8 weeks.

12. The method of claim 10, wherein said treatment lasts for 6 weeks.

13. The method according to one of claims 9-12, wherein said patient is infected with HCV genotype 1.

14. The method according to one of claims 9-12, wherein said patient is infected with HCV genotype 2.

15. The method according to one of claims 9-12, wherein said patient is infected with HCV genotype 3.

16. The method according to one of claims 9-12, wherein said patient is infected with HCV genotype 4.

17. The method according to one of claims 9-12, wherein said patient is infected with HCV genotype 5.

18. The method according to one of claims 9-12, wherein said patient is infected with HCV genotype 6.

19. The method according to one of claims 9-18, wherein said patient is without cirrhosis.

20. The method according to one of claims 9-18, wherein said patient is with compensated cirrhosis.

21. The method according to one of claims 9-18, wherein said patient is a treatment-naive patient.

22. The method according to one of claims 9-18, wherein said patient is a treatment-experienced patient.

23. The pharmaceutical composition according to one of claims 1-8, comprising 80 mg Compound 1.

24. The pharmaceutical composition according to one of claims 1-8, comprising 90 mg Compound 1.

25. The pharmaceutical composition according to one of claims 1-8, comprising 100 mg Compound 1.

26. The pharmaceutical composition according to one of claims 1-8, comprising 60-100 mg Compound 1.

27. The pharmaceutical composition according to one of claims 1-8, comprising 70, 75, 80, 85, 90, 95 or 100 mg Compound 1.

28. The method according to one of claims 9-22, wherein Compound 1 is administered 80 mg once daily.

29. The method according to one of claims 9-22, wherein Compound 1 is administered 90 mg once daily.

30. The method according to one of claims 9-22, wherein Compound 1 is administered 100 mg once daily.

31. The method according to one of claims 9-22, wherein Compound 1 is administered 60-100 mg once daily.

32. The method according to one of claims 9-22, wherein Compound 1 is administered 70, 75, 80, 85, 90, 95 or 100 mg once daily.

33. The pharmaceutical composition according to one of claims 1-8 and 23-27, further comprising glecaprevir.

34. The method according to one of claims 9-22 and 28-32, wherein said patient has end stage renal disease.

35. The method according to one of claims 9-22 and 28-32, wherein said patient has severe renal impairment.
